(19) United States
(12) Patent Application Publication
(10) Pub. No.: US 2025/0282796 A1
(43) Pub. Date: Sep. 11, 2025

(54) PENEM ANTIBACTERIALS AGAINST MYCOBACTERIAL PATHOGENS AND D,D- AND L,D TRANSPEPTIDASES

(71) Applicant: The Johns Hopkins University, Baltimore, MD (US)

(72) Inventors: Craig A. TOWNSEND, Baltimore, MD (US); Hunter R. BATCHELDER, Baltimore, MD (US)

(21) Appl. No.: 18/861,291

(22) PCT Filed: Jun. 1, 2023
(86) PCT No.: PCT/US2023/024103

§ 371 Date: Oct. 29, 2024

Related U.S. Application Data
(60) Provisional application No. 63/347,636, filed on Jun. 1, 2022.

(51) Int. Cl. C07D 499/887; A61K 31/431; A61K 31/541; A61K 31/551; A61P 31/04; A61P 31/06

(52) U.S. Cl. CPC C07D 499/887; A61K 31/431; A61K 31/541; A61K 31/551; A61P 31/04; A61P 31/06

(57) ABSTRACT

A library of penem antimicrobial compounds and their activity against M. tuberculosis and M. abscessus is disclosed.

### FEDERALLY SPONSORED RESEARCH OR DEVELOPMENT

This invention was made with government support under grant AI137329 awarded by the National Institutes of Health. The government has certain rights in the invention.

### BACKGROUND

Tuberculosis (TB), the disease state of Mycobacterium tuberculosis (Mtb) infection, is one of deadliest infectious diseases. Each year an estimated 10 million people fall ill to TB and 1.5 million die from the disease. WHO, 2016. The standard-of-care treatment for drug-susceptible TB involves the administration of four antibiotics for two months followed by two antibiotics for another four months. Additionally, the emergence of multidrug-resistant (MDR), extensively drug-resistant (XDR), and totally drug-resistant (TDR) strains of Mtb have placed an ever growing importance on developing new treatments for TB. Migliori et al., 2020. Moreover, non-tuberculosis mycobacteria (NTM) show strong intrinsic resistance to a broad spectrum of antibiotics. One of the most difficult-to-treat NTM diseases is one caused by Mycobacteroides abscessus (Mab), which is associated with a cure rate as low as 30-50%. Diel et al., 2017; Jarand et al., 2011. Mab pulmonary infections require multidrug therapy that lasts 12-18 months, but treatment is often cut short due to drug toxicity. As there are no FDA-approved antibiotics to treat Mab disease, current treatment recommendations rely on antibiotics approved for other indications. The recommended regimes include an induction phase of at least two months with 3-4 antibiotics, typically including amikacin and a β-lactam, imipenem or cefoxitin. Daley et al., 2020. The limited success of such treatments, despite its length, demonstrates the pressing need for more effective treatments to fight Mtb and NTM infections.

### SUMMARY

In some aspects, the presently disclosed subject matter provides a compound of formula (I):

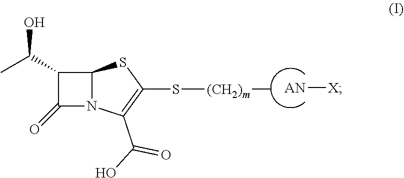

m is an integer selected from 0, 1, 2, 3, and 4;
A is a substituted or unsubstituted nitrogen-containing heterocyclic saturated ring;
X is selected from H, an alkylene chain, and a substituted or unsubstituted heterocyclic ring; and
stereoisomers, pharmaceutically acceptable salts thereof.

In certain aspects, the compound of formula (I) comprises a compound of formula (I′):

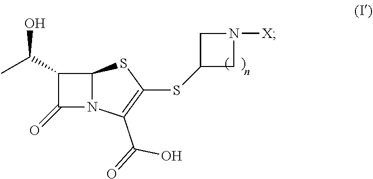

wherein n is an integer selected from 1, 2, and 3.

In particular aspects, A is selected from azetidinyl, pyrrolidinyl, and piperidinyl.

In particular aspects, X is selected from:

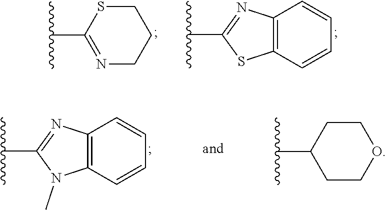

In more particular aspects, the compound of formula (I) is selected from:

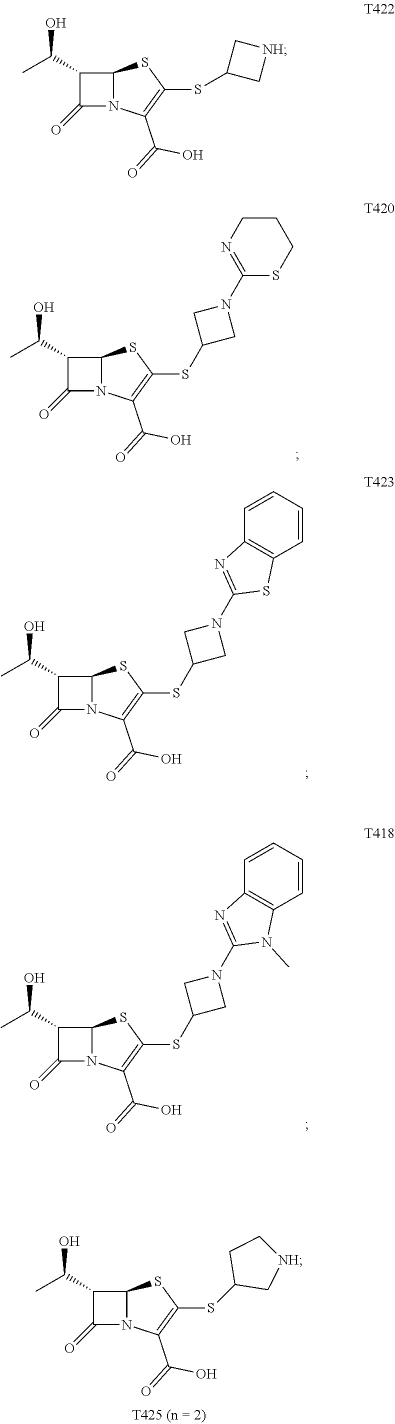 [SMILES: C[C@@H](O)[C@H]1C(=O)N2C(C(=O)O)=C(SC3CNC3)S[C@H]12 ; C[C@@H](O)[C@H]1C(=O)N2C(C(=O)O)=C(SC3CN(C4=NCCCS4)C3)S[C@H]12 ; C[C@@H](O)[C@H]1C(=O)N2C(C(=O)O)=C(SC3CN(c4nc5ccccc5s4)C3)S[C@H]12 ; C[C@@H](O)[C@H]1C(=O)N2C(C(=O)O)=C(SC3CN(c4nc5ccccc5n4C)C3)S[C@H]12 ; C[C@@H](O)[C@H]1C(=O)N2C(C(=O)O)=C(SC3CCNC3)S[C@H]12]

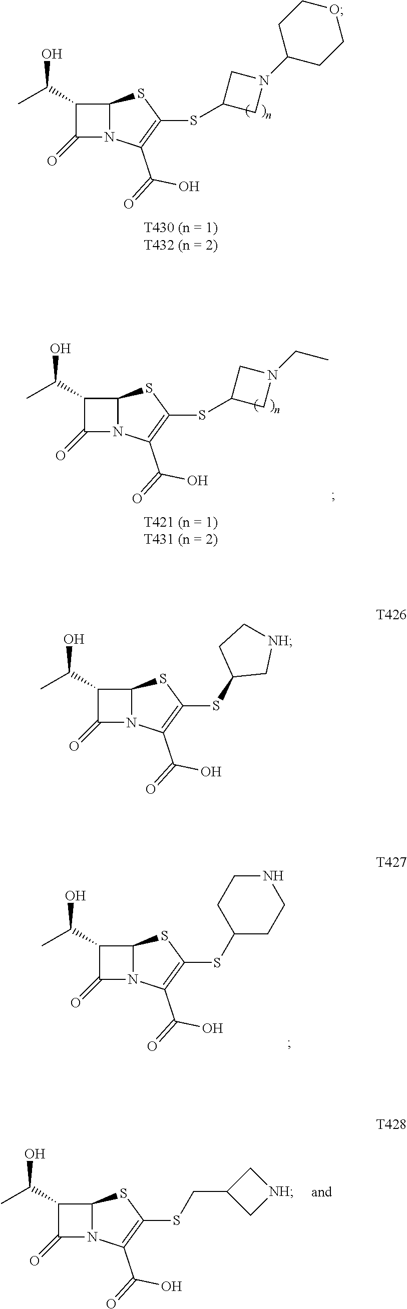

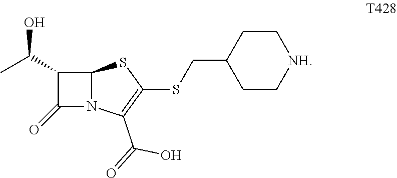 [SMILES: C[C@@H](O)[C@H]1C(=O)N2C(C(=O)O)=C(SCC3CCNCC3)S[C@H]12]

In other aspects, the presently disclosed subject matter provides a method for treating a mycobacterial infection in a subject, the method comprising administering a subject in need of treatment thereof, a therapeutically effective amount of a compound of formula (I).

In certain aspects, the mycobacterial infection is selected from a Mycobacterium tuberculosis infection, a nontuberculous mycobacterial infection, and a Mycobacteroides abscessus infection.

In certain aspects, the method further comprises administering to the subject one or more antibiotics in combination with the compound of formula (I).

In particular aspects, the one or more antibiotics comprise one or more β-lactamase inhibitors. In more particular aspects, the one or more β-lactamase inhibitors is selected from clavulanate, sulbactam, tazobactam, and avibactam. In yet more particular aspects, the one or more β-lactamase inhibitors comprises clavulanate.

In other aspects, the presently disclosed subject matter provides a composition comprising a compound of formula (I) and one or more antibiotics. In particular aspects, the one or more antibiotics comprise one or more β-lactamase inhibitors. In more particular aspects, the one or more β-lactamase inhibitors is selected from clavulanate, sulbactam, tazobactam, and avibactam. In yet more particular aspects, the one or more β-lactamase inhibitors comprises clavulanate.

In other aspects, the presently disclosed subject matter provides a method for inhibiting an L,D-transpeptidase (Ldt) or a D,D-transpeptidase, the method comprising contacting the L,D-transpeptidase (Ldt) or D,D-transpeptidase with a compound of formula (I) In some aspects, the contacting is in vitro or in vivo.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows that Ldt_{Mt2} is acylated by all experimental penems tested, with each forming identical M+86 fragments. Single blue dot represents apo-Ldt_{Mt2}, m/z=38,086 Da, and two blue dots represents the Ldt_{Mt2}-penem fragment adduct, 38,172 Da; and

FIG. 2A, FIG. 2B, FIG. 2C, and FIG. 2D demonstrate that intact-protein UPLC-HRMS analysis demonstrates differential stability of select DacB2-penem adducts after washing away free drug and incubating for 24 h. (FIG. 2A) UPLC-HRMS analysis of apo-DacB2 one square, 27435 Da). (FIG. 2B) Meropenem fully reacts with DacB2 and the DacB2-meropenem adduct (decarboxylated, one circle, 27,774 Da; intact, two circles, 27,818 Da) is stable after removal of drug for 24 h. (FIG. 2C) Like meropenem, T422 and T426 form adducts that are stable for at least 24 h after drug removal, while T405 and T428 exhibit loss of 24% and 14% of drug, respectively. (FIG. 2D) Mass spectra of T405 (decarboxylated, one triangle, 27,778 Da, intact, two triangles 27,822 Da), T422 (decarboxylated, one star, 28033 Da; intact, two stars, 28,077 Da), T426 (decarboxylated, one cross, 27,708 Da; intact, two crosses, 27,752 Da), and T428 (decarboxylated, one diamonds, 27,708 Da; intact, two diamonds, 27,752 Da) adducts of DacB2 before and after washout. One black square represents apo-DacB2 (27,435 Da).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Compounds of Formula (I)

In some embodiments, the presently disclosed subject matter provides a compound of formula (I):

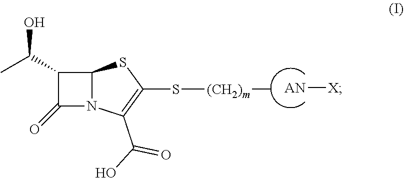

m is an integer selected from 0, 1, 2, 3, and 4;
A is a substituted or unsubstituted nitrogen-containing heterocyclic saturated ring;
X is selected from H, an alkylene chain, and a substituted or unsubstituted heterocyclic ring; and stereoisomers, pharmaceutically acceptable salts thereof.

In certain embodiments, the compound of formula (I) comprises a compound of formula (I′):

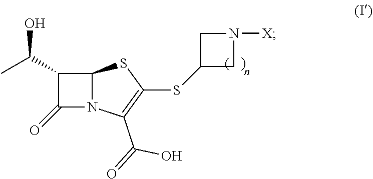

wherein n is an integer selected from 1, 2, and 3.

In particular embodiments, A is selected from azetidinyl, pyrrolidinyl, and piperidinyl.

In particular embodiments, X is selected from:

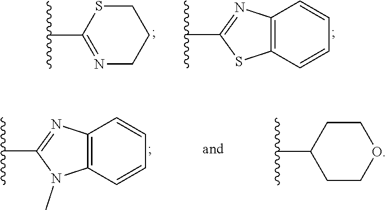

In more particular embodiments, the compound of formula (I) is selected from:

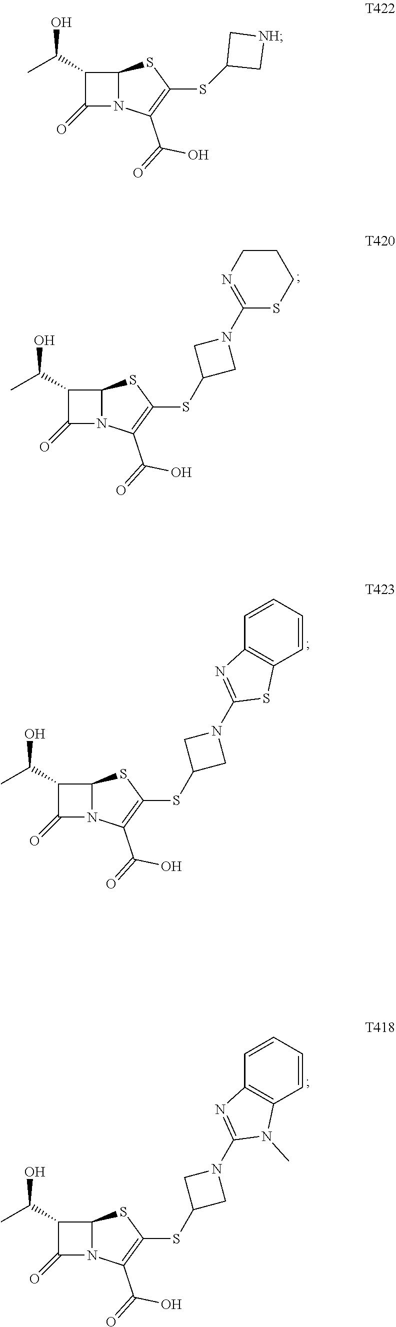 [SMILES: C[C@@H](O)[C@H]1C(=O)N2C(C(=O)O)=C(SC3CNC3)S[C@H]12 ; C[C@@H](O)[C@H]1C(=O)N2C(C(=O)O)=C(SC3CN(C4=NCCCS4)C3)S[C@H]12 ; C[C@@H](O)[C@H]1C(=O)N2C(C(=O)O)=C(SC3CN(c4nc5ccccc5s4)C3)S[C@H]12 ; C[C@@H](O)[C@H]1C(=O)N2C(C(=O)O)=C(SC3CN(c4nc5ccccc5n4C)C3)S[C@H]12]

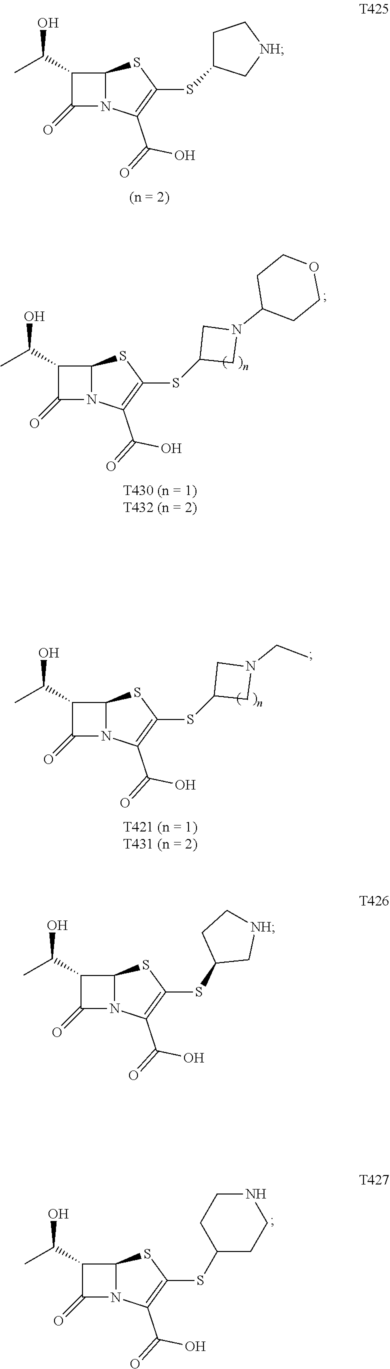

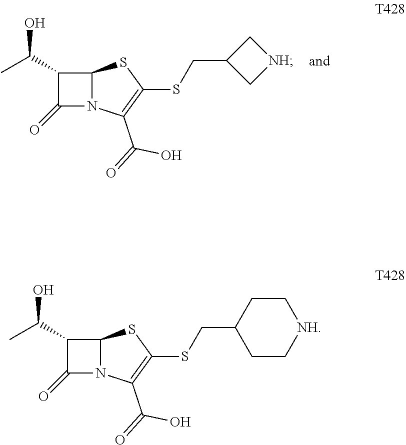 [SMILES: C[C@@H](O)[C@H]1C(=O)N2C(C(=O)O)=C(SCC3CNC3)S[C@H]12 ; C[C@@H](O)[C@H]1C(=O)N2C(C(=O)O)=C(SCC3CCNCC3)S[C@H]12]

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to the presently disclosed penem antimicrobial agents are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

Generally, a “heterocyclic compound” is a cyclic compound that comprises atoms of at least two different elements as members of its ring. For example, a nitrogen-containing heterocyclic ring comprises at least one nitrogen atom and at least one other atom. In some embodiments, a nitrogen-containing heterocyclic ring comprises at least one nitrogen atom, wherein the remainder of the atoms are carbon.

A “saturated heterocyclic ring” includes carbon atoms having only single bonds and the maximum number of hydrogen atoms on each carbon atom.

Representative nitrogen-containing heterocyclic saturated rings include aziridine (3-member nitrogen-containing ring); azetidine (4-member nitrogen-containing ring); pyrolidine (5-member nitrogen-containing ring); and piperidine (6-member nitrogen-containing ring).

An “alkylene chain” refers to an alkanediyl group, i.e., a divalent radical of the general formula CₙH₂ₙ, derived from an aliphatic hydrocarbon. Representative alkylene groups can have from 1 to about 20 carbon atoms, e.g., 1, 2, 3, 4, 5, 6, 7, 8, 9, 10, 11, 12, 13, 14, 15, 16, 17, 18, 19, or 20 carbon atoms. Exemplary alkylene groups include methylene (—CH₂—); ethylene (—CH₂—CH₂—); propylene (—(CH₂)₃—); and the like.

As used herein, “heterocyclic rings” include a ring structure having from one to three heteroatoms independently selected from oxygen, sulfur, and nitrogen, Representative heterocyclic ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, tetrahydropyranyl, and the like. More particularly, a heterocyclic ring system and include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, benzo[d]thiazolyl, purinyl, 2-benzimidazolyl, 5-indolyl, 1-isoquinolyl, 5-isoquinolyl, 2-quinoxalinyl, 5-quinoxalinyl, 3-quinolyl, 6-quinolyl, 5,6-dihydro-4H-1,3-thiazinyl, 1-methyl-1H-benzo[d]imidazole, and the like.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

The symbol ( [symbol] ) denotes the point of attachment of a moiety to the remainder of the molecule.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

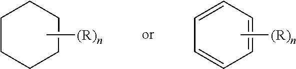

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

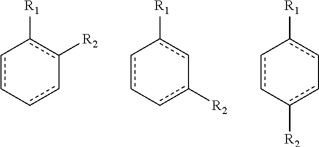

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

Unless otherwise explicitly defined, a “substituent group,” as used herein, includes a functional group selected from one or more of the following moieties, which are defined herein:

The term “alkyl,” by itself or as part of another substituent, means, unless otherwise stated, a univalent group derived from an alkane by removal of a hydrogen atom from any carbon atom —CₙH₂ₙ₊. Representative alkyl groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, and dodecyl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R″′ are each independently selected from the group consisting of alkyl groups. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described.

“Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

### B. Methods for Treating a Mycobacterial Infection

In other embodiments, the presently disclosed subject matter provides a method for treating a mycobacterial infection in a subject, the method comprising administering a subject in need of treatment thereof, a therapeutically effective amount of a compound of formula (I).

In certain embodiments, the mycobacterial infection is selected from a Mycobacterium tuberculosis infection, a nontuberculous mycobacterial infection, and a Mycobacteroides abscessus infection.

In certain embodiments, the method further comprises administering to the subject one or more antibiotics in combination with the compound of formula (I).

In particular embodiments, the one or more antibiotics comprise one or more β-lactamase inhibitors. In more particular embodiments, the one or more β-lactamase inhibitors is selected from clavulanate, sulbactam, tazobactam, and avibactam. In yet more particular embodiments, the one or more β-lactamase inhibitors comprises clavulanate.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In other embodiments, the presently disclosed subject matter provides a method for inhibiting an L,D-transpeptidase (Ldt) or a D,D-transpeptidase, the method comprising contacting the L,D-transpeptidase (Ldt) or D,D-transpeptidase with a compound of formula (I) In some embodiments, the contacting is in vitro or in vivo.

### C. Pharmaceutical Compositions

In other embodiments, the presently disclosed subject matter provides a pharmaceutically composition comprising a compound of formula (I) and one or more antibiotics. In particular embodiments, the one or more antibiotics comprise one or more β-lactamase inhibitors. In more particular embodiments, the one or more β-lactamase inhibitors is selected from clavulanate, sulbactam, tazobactam, and avibactam. In yet more particular embodiments, the one or more β-lactamase inhibitors comprises clavulanate.

The present disclosure further provides a pharmaceutical composition including a compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In certain embodiments, the compound of formula (I) or a pharmaceutical composition thereof is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick. As used herein, the term administrating via an “intranasal route” refers to administering by way of the nasal structures.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Structure-Activity Relationship of Penem Antibiotic Sidechains for Use Against Mycobacteria Reveal Highly Active Compounds

### 1.1 Overview

The rise of antibiotic-resistant Mycobacterium tuberculosis and nontuberculous mycobacterial infections has placed an ever-increasing importance on discovering new antibiotics to treat these diseases. Recently, a new penem, T405, was discovered to have strong antimicrobial activity against Mycobacterium tuberculosis and Mycobacteroides abscessus. Here a penem library of sidechain variants was synthesized and its antimicrobial activity evaluated against M. tuberculosis H₃Rv and M. abscessus ATCC 19977. Several new penems with antimicrobial activity stronger than the standard-of-care carbapenem antibiotics were identified with some candidates improving on the activity of the lead compound, T405. The penems with the strongest activity identified in this study were then biochemically characterized in reaction with the representative L,D-transpeptidase Ldt_{Mt2} and the representative penicillin-binding protein, D,D-carboxypeptidase DacB2.

### 1.2 Background

The β-lactam class of antibiotics has been hugely successful at treating a broad spectrum of bacterial diseases making up 50% of all prescribed antibiotics. Hamad, 2010. Historically, β-lactams were considered ineffective against mycobacteria, Robinson, 1943; Smith and Emmart, 1944, but more recently there has been renewed interest in their use to treat mycobacterial infections. Storey-Roller and Lamichhane, 2018. Of the five sub-classes of β-lactam antibiotics, carbapenems and penems stand out as the most potent against mycobacteria. Dhar et al., 2015; Batchelder et al., 2020; Hugonnet et al., 2009; Tiberi et al., 2016. Unsurprisingly, these two classes are very similar with the only structural difference between the pharmacophores being the presence of a sulfur in the penem at position 1 of the bicyclic ring instead of a methylene in carbapenems (Scheme 1). Carbapenems have been heavily explored with multiple drugs on the market, while penems are only represented commercially by faropenem. Meropenem, a member of the carbapenem subclass, in combination with clavulanate, a β-lactamase inhibitor, has unmistakable bactericidal activity in the sputum of TB patients and has been used effectively in the clinic for treating multidrug-resistant TB infections. Hugonnet et al., 2009; Tiberi et al., 2016; Diacon et al., 2016; De Jager et al., 2022.

These results have spurred interest in developing new carbapenems against Mtb. Iannazo et al., 2016; Saidjalolov et al., 2021. The recent development of atypical carbapenems has shown promising progress against Mtb and Mab. Gupta et al., 2021. Additionally, faropenem and the newly developed penem T405 have displayed potent activity against Mtb and Mab. Dhar et al., 2015; Batchelder et al., 2020. While carbapenems have been the main focus in β-lactam development against TB, penems remain relatively unexplored.

The mechanism of action of β-lactam antibiotics takes place through the inhibition of essential enzymes in the biosynthesis of the organism's cell wall to block completion of cell wall crosslinking steps. Kitano and Tomasz, 1979. As a result, mounting turgor pressure ruptures the cell. Mtb and NTMs are diverse in their peptidoglycan structures and synthesis and, consequently, have atypical peptidoglycan. Lavollay et al., 2008; Gupta et al., 2010. There are two classes of enzymes in mycobacteria that β-lactams must inhibit to be successful antibiotics, the penicillin binding proteins (PBPs) and the L,D-transpeptidases (Ldt). The PBP enzyme class contains the D,D-transpeptidases, which are responsible for the formation of the classical 4-3 peptidoglycan cross linkages, and the homologous D,D-carboxypeptidases, which are responsible for generation of the tetrapeptide substrates of Ldts. This class of enzymes utilizes an active site serine nucleophile to attack into the D-Ala-D-Ala peptide bond generating an acyl-enzyme intermediate. The β-lactams utilize this native reactivity by lending their strained amide to be attacked, in turn forming a stable ester-bound intermediate that inhibits the PBP from further reaction. Blumberg and Strominger, 1974. Additionally, there are the Ldts, which catalyze peptidoglycan 3-3 crosslinks between tetra- and pentapeptide stems. These linkages predominate in stationary phase Mtb and Mab. Lavollay et al., 2008; Lavollay et al., 2011. The evolutionarily distinct Ldt class utilizes a cysteine nucleophile to perform the transpeptidase reaction, and consequently, when acylated by β-lactams, a thioester linkage results. Erdemli et al., 2012. The thioester linkage catalyzed by Ldts is more labile compared to the oxyester linkage of PBPs resulting in an intrinsic β-lactam resistance of the Ldt enzyme class. Lavollay et al., 2008; Lavollay et al., 2011. Carbapenems and penems are the β-lactams that most effectively inhibit both of these classes of enzymes.²⁵⁻³¹ Kim et al., 2013; Dubde et al., 2012; Cordillot et al., 2013; Kumar et al., 2017a; Steiner et al., 2017; Kumar et al., 2017b; Bianchet et al., 2017.

Undoubtedly the largest contributor to Mtb resistance against β-lactam antibiotics is the presence of the highly active class A Ambler β-lactamase, BlaC and select PBPs. Wang et al., 2006; Kumar et al., 2022. BlaC efficiently hydrolyzes nearly all classes of β-lactams. Tremblay et al., 2010. Notably, however, some carbapenems are only turned over slowly by BlaC resulting in an inherent advantage compared to other β-lactams. Hugonnet et al., 2009; Tremblay et al., 2010. In combination with clavulanate, a well-established β-lactamase inhibitor, many β-lactams regain activity against Mtb. Hugonnet et al., 2009.

A new penem, T405, was identified from an antibiotic screen against Mtb and showed strong antimicrobial activity against laboratory strains of Mtb and Mab, as well as a panel of 20 clinical isolates of Mab. Batchelder et al., 2020. While T405 utilizes the penem pharmacophore, the sidechain branching from C2 gives the drug its unique properties (Scheme 1). The sidechain is composed of the azetidine A-ring and the dihydrothiazole B-ring (Scheme 1). To better understand the influence of the sidechain on the activity of T405 and in the hope to generate more potent antibiotics against Mtb and Mab, modifications to these sidechain rings were designed and synthesized. These rings were separately modified to generate a library to probe the influence of both rings on antibiotic activity. The structure-activity relationship (SAR) of these newly generated penems was examined by in vitro antimicrobial activity assays. Moreover, the most potent compounds were further examined in in vitro inhibition assays with the D,D-carboxypeptidase and PBP representative DacB2 and the Ldt representative Ldt_{Mt2}.

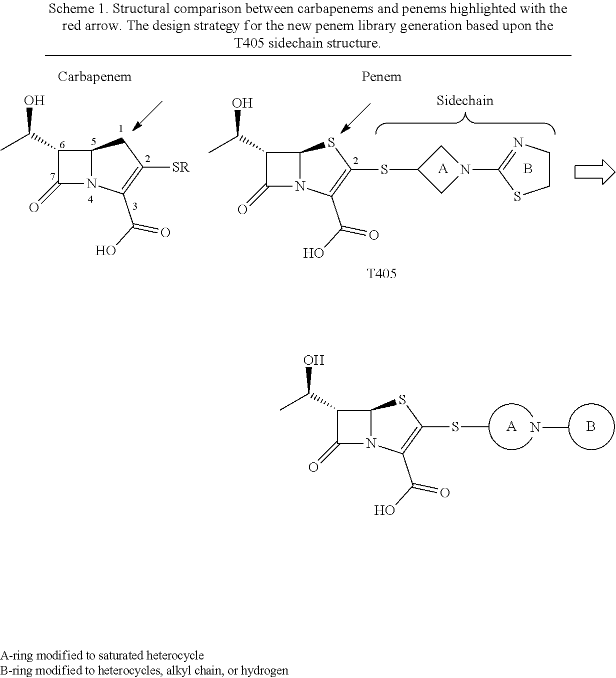

### 1.3 Results

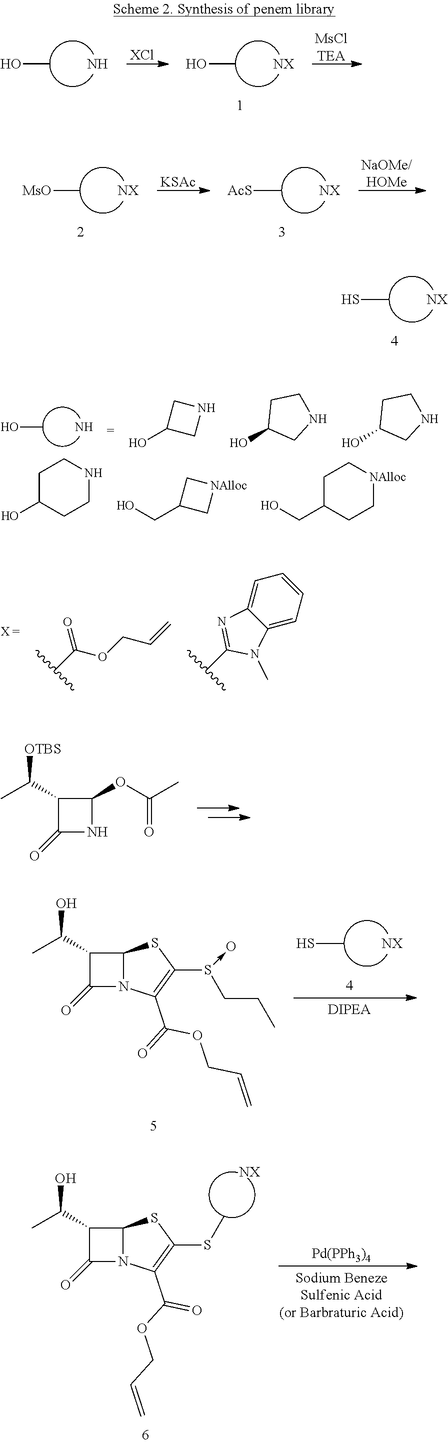

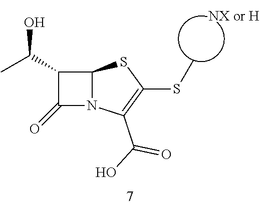 [SMILES: "NX or H" ; CSC1=C(C(=O)O)N2C(=O)[C@H]([C@@H](C)O)[C@H]2S1]

### 1.3.1 Chemistry

To elucidate the effect of the C2 sidechain on T405 antimicrobial activity, a variety of penems with different sidechains was synthesized. The sidechains varied in two sections denoted as the A-ring and the B-ring (Scheme 1). The sidechains were synthesized starting from an amino alcohol. The nitrogen of heterocycle A was first either protected as its allyl carbamate (alloc) via reaction with allyl chloroformate, or coupled with 2-chloro benzimidazole (denoted by X in Scheme 2). Once the reactive nitrogen was masked, the alcohol was activated as the methanesulfonate, 2, by reaction with methanesulfonyl chloride. The protected thiol was then installed through substitution of the activated alcohol with potassium thioacetate to yield 3. Lastly, the thioacetate was reacted with sodium methoxide to reveal the deprotected sidechain thiol, 4.

The penem core was prepared as previously described to give access to compound 5. Batchelder et al., 2020. By employing a convergent strategy to the synthesis, the sidechains could be incorporated at a late stage into the oxidized penem core, 5, through a β-addition/elimination reaction. Once the thioether sidechain was installed, global allyl deprotection efficiently afforded the deprotected carboxylic acid (T418) and the secondary nitrogen if applicable (T422, T425, T426, T427, T428, and T429; for structures refer to Table 1). The secondary nitrogen could then be further derivatized through reductive amination (T421, T430, T431, and T432) or by reaction with thioisocyanates followed by in situ intermolecular cyclization (T423 and T420) (Scheme 3).

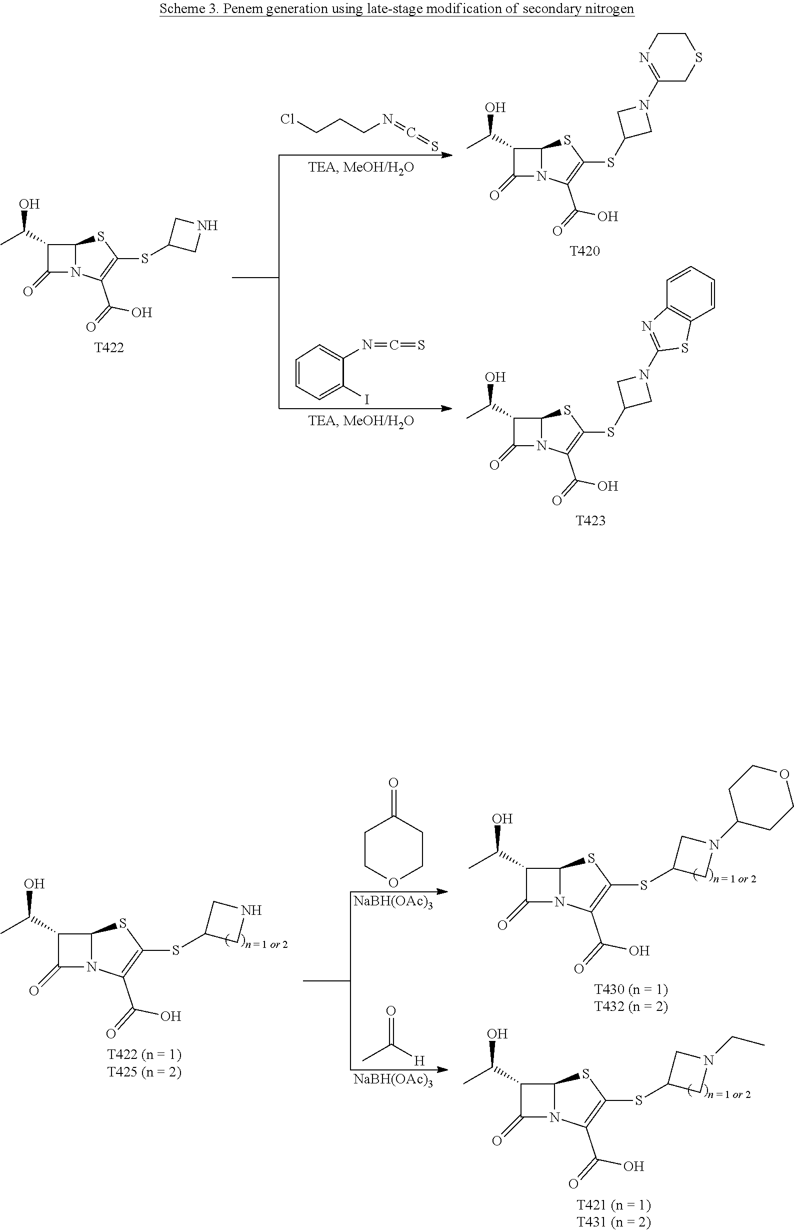

TABLE 1
MIC (μg/mL) of penem library against Mab ATCC 19977, M.tb H37Rv/clav,
M.tb HN878/clav, M. av 101, and M.av 104.

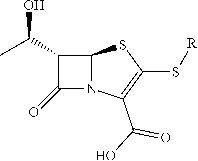

MIC (μg/ml)

| | | | Mtb H37Rv with | |
|---|---|---|---|---|
| | | | Clavulanate | Mab ATCC |
| Compound | R: | Mtb H37Rv | (5 μg/ml) | 19977 |
| T418 | 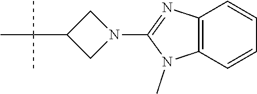 | 2 | 0.5 | 64 |
| T423 | 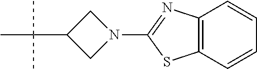 | 8 | 2 | 64 |
| T430 | 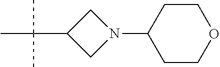 | 4 | ND | 4 |
| T420 | 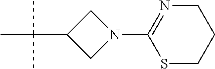 | 2 | 0.5 | 16 |
| T405 | 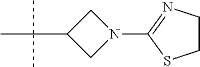 | 0.5 | 0.5 | 2 |
| T421 | 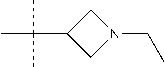 | 1 | 0.5 | 4 |
| T422 | 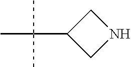 | 0.5 | 0.5 | 4 |
| T432 | 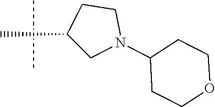 | 4 | ND | 4 |
| T431 | 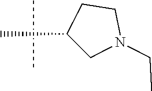 | 4 | ND | 4 |
| T425 | 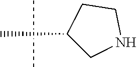 | 0.5 | 0.25 | 2 |
| T426 | 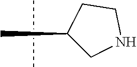 | 0.5 | 0.25 | 2 |
| T427 | 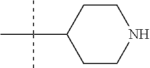 | 1 | 0.5 | 4 |
| T428 | 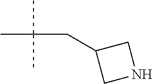 | 0.5 | 0.25 | 8 |
| T429 | 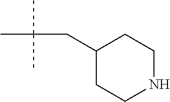 | 0.5 | 0.25 | 4 |
| Imipenem | | | | 8 |
| Meropenem | | 8 | 0.5 | |

### 1.3.2 Activity Against Mtb

The minimum inhibition concentration (MIC) was determined against Mtb H₃Rv with and without the addition of the β-lactamase inhibitor clavulanate (Table 1). Initial SAR investigation explored modifications of the B-ring of T405 while maintaining the azetidine A-ring. While increasing the B-ring from five members to six in T420 increased the MIC by two dilutions, activity was recovered in the presence of clavulanate. The N-methyl benzimidazole B-ring of T418, increased the MIC by four-fold although it showed the strongest activity of the compounds containing an aromatic ring. In a continuing trend the benzothiazole of T423 increased the MIC 16-fold.

Truncation of the B-ring of T405 to yield just the terminal azetidine T422 showed the same activity as T405. To further explore the role of the A-ring on the antimicrobial activity, rings of increasing sizes were synthesized while maintaining the secondary nitrogen of the truncated B-ring. Increasing the A-ring size to five members in T425 and T426 produced the same MIC as T405. Moreover, the addition of clavulanate lowered the MIC of both compounds two-fold to 0.25 μg/mL. There was no stereochemical preference in activity between the two diastereomers T425 and T426. Upon increasing the A-ring to six members in T427, the MIC increased two-fold. By extending the six-membered ring from the penem core by a methylene in T429, however, the activity was recovered. Lastly, by extending T422 by a methylene, T428, the MIC in the presence of clavulanate improved by two-fold as well. Adding an ethyl or adding a tetrahydropyran to T422 to form T421 or T430, as well as to T425 to from T431 and T432, respectively, only decreased the antimicrobial activity compared to the secondary amine.

### 1.3.3 Activity Against Mab

The in vitro antibacterial activity of the penem library was tested against the Mab strain ATCC 19977. It was previously shown that T405 possesses strong antimicrobial activity against this laboratory strain as well as 20 clinical isolates of the Mab complex. Batchelder et al., 2020. Much like in Mtb, increasing the size of the B-ring of T405 to a six-member ring in T420 had a negative effect on the activity against Mab. Moreover, introduction of any aromatic ring at this position had a detrimental effect on the activity against Mab as shown by T418, and T423. Removal of the B-ring of T405 resulted in 2-fold weaker activity for T422. When the size of the A-ring was changed to a five-membered ring, however, the MIC was restored to that of T405, 2.0 μg/mL. When the size of the A-ring was increased again and/or spaced from the penem core by a methylene, the activity was reduced again by two-fold or more.

1.3.4 Penem Reactions with Ldt_{Mt2}

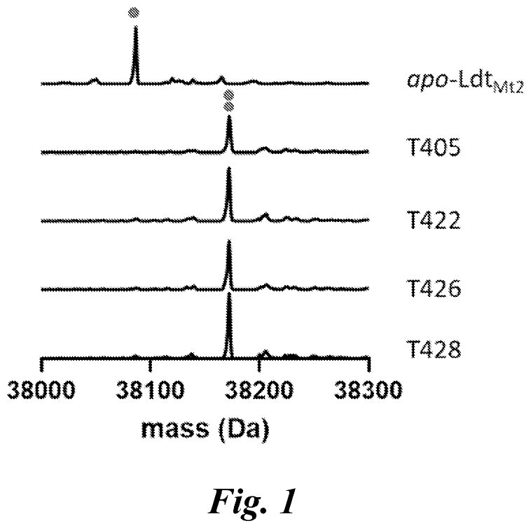

A group of penems, T405, T422, T426, and T428, which had the greatest antimicrobial activity was reacted with Ldt_{Mt2}. The resulting covalent drug adduct was measured by intact-protein UPLC-HRMS analysis. All penems tested formed a +86 Da Ldt_{Mt2} adduct (FIG. 1), owing to characteristic scission of the C₅-C₆ bond, a process that has been reported previously for the penem, faropenem, Kumar et al., 2017a; Lohans et al., 2019, and C5-substituted carbapenems. Gupta et al., 2021. Notably, fragmentation to the +86 Da adduct is irreversible, unlike the related reversible carbapenem adducts of Ldt_{Mt2}, Zandi et al., 2021, and its hydrolytic stability has been previously characterized. Dhar et al., 2015.

1.3.5 Penem Reactions with DacB2

The D,D-carboxypeptidase DacB2 is a well-studied exemplar of the broader PBP enzyme class, and its activity is required to precede the function of Ldts. Kumar et al., 2012. We thus analyzed covalent inhibition of DacB2 by select penems to gain insight into the best inhibitor(s) of DacB2 and potential inhibition efficiency for PBPs in general. The adduct off-rate, or adduct stability, directly relates to enzyme occupancy. For slow inhibitors, such as carbapenems, the adduct stability has been shown to be more correlated with in vivo activity than adduct on-rate. Walkup et al., 2015; Copeland et al., 2006; Lu et al., 2010.

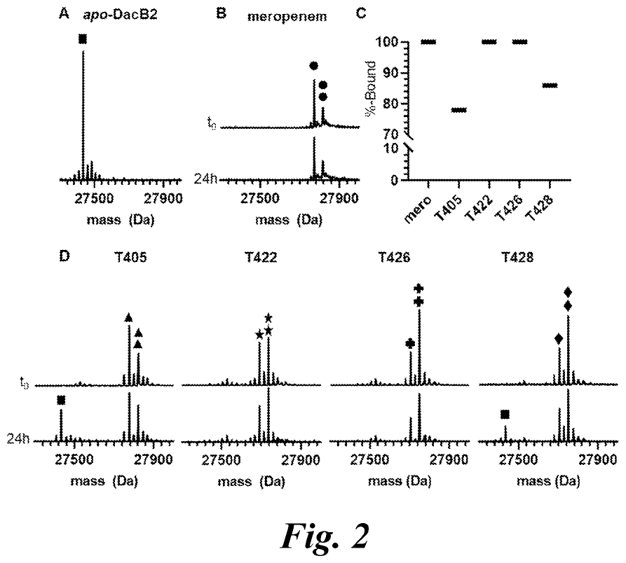

To measure the adduct stability, DacB2 and each penem were reacted, the resultant DacB2-penem adduct was washed free of drug, and extent of residual binding was measured by the appearance of apo-DacB2 (FIG. 2A) after an overnight incubation. As a point of comparison, the same experiment was performed with the clinically used carbapenem, meropenem (FIG. 2B). We found that DacB2-T405 and DacB2-T428 exhibited some loss of adduct over time, retaining 76% and 86% of inhibitor, respectively, after 24 h while DacB2-T422, DacB2-T426, as well as DacB2-meropenem remained completely bound over this period of time (FIG. 2C and FIG. 2D).

### 1.4 Discussion

An SAR database of penems was prepared and used to investigate the influence of the T405 C2 sidechain on antimicrobial activity against Mtb H₃₇Rv and Mab ATCC 19977. To examine this question the C2 sidechain was varied at two sites, the A-ring and the B-ring (Scheme 1). This SAR study resulted in five compounds having activities comparable to T405 against Mtb and 15 compounds with better or equivalent activity compared to meropenem, the carbapenem recommended for use against MDR-TB. Clavulanate is commonly administered with carbapenems to inhibit BlaC. This supplementation has a large effect whereby the MIC of meropenem shifts from 8 μg/mL without clavulanate to 0.5 μg/mL with it. Hugonnet et al., 2009. In contrast, several penem antibiotics that already possessed low MICs did not show such a large response, if any, to addition of the 3-lactamase inhibitor. This difference would indicate that either the penems are not significantly hydrolyzed by BlaC or that the limit of the MICs is not dependent on the rate of this hydrolysis. Therefore, this group of compounds has potential for use as a sole agent treatment as opposed to requiring combination therapy with clavulanate.

The penem library was also tested againstMab ATCC 19977 that is intrinsically resistant to several antibiotics. Many of the same trends in the SAR data were borne out between the two mycobacteria. Most notably, any aromatic ring in the C2 side chain drastically reduced the activity. Additionally, many sidechains produced good activity againstMab, with MICs averaging around 4 μg/mL. Only the five-member ring penems, T425 and T426, had activity equal to T405 against Mab (MIC of 1 g/mL). By way of comparison, the carbapenem imipenem, a first-line drug in the treatment of Mab infection has an MIC of 8 μg/mL. Eleven penems from the library showed equal or lower MIC compared to imipenem.

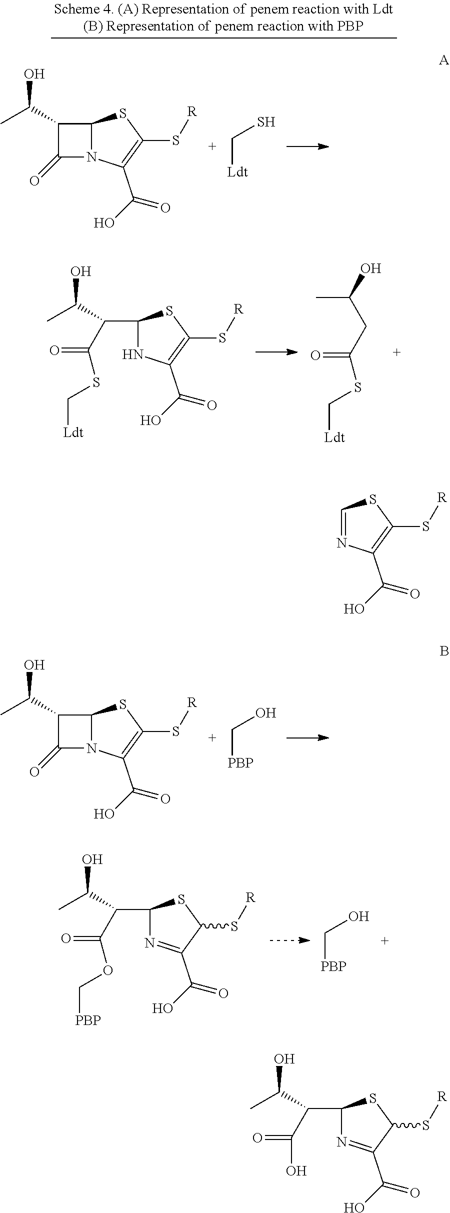

Of the three classes of β-lactam targets known to confer antibacterial activity, namely, Ldts, PBPs, and β-lactamases, representative Ldts and PBPs were selected for target engagement assays with select penems. As the β-lactamase inhibitor clavulanate has minimal effect on the MIC of these penems, BlaC is likely not responsible for differential activity and was not separately evaluated in enzyme inhibition assays. The adduct formation and stability of the most active penems, T422, T426, and T428, was measured against Ldt_{Mt2} and compared to the penem T405. When adduct formation was monitored with Ldt_{Mt2}, in-solution fragmentation to a M+86 Da 3-hydroxybutyryl group ensued for each penem (Scheme 3A). This observation agrees with previous studies of the faropenem adduct with Ldts. Kumar et al., 2017a; Lohans et al., 2019. Moreover, the data show that the penem breakdown on Ldt_{Mt2} is not dependent on the identity of the sidechain. When these results are combined with the observation that faropenem rapidly forms the same M+86 Da fragment after initial thioester adduct formation to active-site cysteine variants of β-lactamases, Lohans et al., 2019, it is clear that fragmentation of penem thioester adducts is facile and generalizable. When this group of penems was tested against the PBP representative DacB2, each penem formed intact adducts (Scheme 3B). When monitored for 24 hours T422 and T426 were fully bound, as seen for meropenem, while T405 and T428 were partially hydrolyzed. This behavior would indicate that T422 and T426 are better inhibitors of DacB2 because of their longevity bound to the protein. It has been hypothesized that antibiotics with long-lasting enzyme inhibition are particularly desirable to be effective against slow-growing bacterial species such as Mtb that divide approximately every 24 hours, as precedented by D-cycloserine inhibition of Mtb alanine racemase.³⁸ Due to DacB2 stability in vitro, adduct lifetimes were measured at 20° C. as opposed to physiological temperature of 37° C., where chemical processes are faster and drug lifetimes would be correspondingly shorter.

Several penem antibiotics discovered in this study showed activity against Mtb and Mab that was more potent than the respective carbapenem clinical comparators. MIC data from the library show the importance of the A-ring to the activity of the candidates while modifications to the B-ring were shown to be either negative or neutral. Moreover, the in vitro analysis showed that the sidechain has no effect on the adduct stability with Ldts but does affect the adduct stability when reacted with PBPs. This observation suggests the difference in the sidechain activity is more dependent on the resulting inhibition of PBPs. The database generated here sheds light on the role the sidechain plays on the activity of penems against mycobacteria and could have broader applications to other β-lactam molecules. In this process several new, highly active penems have been identified to advance for future studies.

### 1.5 Materials and Methods

### 1.5.1 Chemical Compounds

Penems were synthesized as described in the supplementary information. Imipenem and meropenem were purchased from Carbosynth (San Diego, CA). Clavulanate was purchased from Sigma-Aldrich (St. Louis, MO).

### 1.5.2 Minimum Inhibitory Concentration (MIC) Assay

MICs were determined as previously described using the broth microdilution assay in Middlebrook 7H9 media supplemented with 10% oleic acid, albumin, dextrose and catalase but without Tween 80. Kaushik et al., 2017; Kaushik et al., 2019. Powdered drug stocks were reconstituted in dimethyl sulfoxide and two-fold serial dilutions were prepared in Middlebrook 7119 broth to obtain final drug concentrations in 96-well microtiter plates. Approximately 105 colony forming units (CFU)/mL of bacteria from an exponentially growing culture were added to each well. Mab and Mtb cultured without drug in Middlebrook 7H9 broth alone were included in each plate as positive and negative controls, respectively. Plates were incubated at 30° C. for 72 h and at 37° C. for 14 d for MIC determination against Mab and Mtb, respectively in accordance to Clinical and Laboratory Standards Institute (CLSI) guidelines. CLSI, 2018. Growth or lack thereof was assessed by visual inspection and an MIC for each drug was defined as the lowest concentration that prevented visible growth.

### 1.5.3 Protein Expression and Purification

Ldt_{Mt2} (ΔN55), and DacB2(ΔN27) were expressed and purified as previously described. Basta et al., 2015. Enzyme concentrations were determined using the Beer-Lambert Law by measuring Δ₂₈₀ by ultraviolet-visible spectroscopy in 7 M guanidinium chloride and calculated extinction coefficient for DacB2, Kumar et al., 2017, and 84,000 M⁻¹ cm⁻¹ for Ldt_{Mt2}, previously determined by amino acid analysis. Zandi et al., 2021.

### 1.5.4 UPLC-High Resolution MS

UPLC-high resolution MS (UPLC-HRMS) experiments were analyzed on a Waters Acquity H-Class UPLC system equipped with a multiwavelength UV-Vis diode array detector in conjunction with a Waters Acquity BEH-300 μL UPLC column packed with a C4 stationary phase (2.1×50 mm; 1.7 μm) to analyze intact proteins in tandem with HRMS analysis by a Waters Xevo-G2 quadrupole-time of flight electrospray ionization MS.

### 1.5.5. Intact Protein UPLC-HRMS

Enzyme samples were separated at 60° C. to enhance peak resolution with a flow rate of 0.3 mL/min and the following mobile phase: 0 to 1 min 90% water, 10% ACN, 0.1% formic acid (FA); 1 to 7.5 min gradient up to 20% water, 80% ACN, 0.1% FA; 7.5 to 8.4 min 20% water, 80% ACN, 0.1% FA; 8.4 to 8.5 min linear gradient up to 90% water, 10% ACN, 0.1% FA; 8.5 to 10 min 90% water+10% ACN, 0.1% FA. The first minute of eluate was discarded to remove salts and buffer online. Samples were analyzed in positive mode and deconvoluted from m/z distributions into neutral masses using the Maxent1 algorithm within Masslynx. Data were then normalized to the sum of intensities and plotted within Prism 9.3.0.

### 1.5.6 Ldt_{Mt2} Adduct Formation

Each penem (20 μM) was incubated separately with Ldt_{Mt2} (2 M) for 1 h in 25 mM HEPES pH 7.0 buffer at 20° C. Samples were then subjected to intact protein UPLC-HRMS analysis for determination of adduct formation.

### 1.5.7 DacB2 Adduct Stability Comparison

Penems and meropenem (20 M) were incubated individually with DacB2 (2 M) for 1 h in 25 mM HEPES pH 7.0 buffer at 20° C. Complete adduct formation was confirmed by intact protein UPLC-HRMS analysis at which point excess (carba)penem was removed by two sequential buffer exchanges with Thermo-Scientific Zeba 7 kDa spin desalting columns which had been pre-equilibrated with 25 mM HEPES pH 7.0 buffer. After 24 h, DacB2 was again subjected to intact protein UPLC-HRMS analysis. Percent bound was determined in Biopharmalynx 1.3.2 by summing ion counts of intact and decarboxylated forms of DacB2-adducts, and dividing by the sum of apo and bound form ion counts, as described previously. Zandi et al., 2021.

### Example 2

### Experimental Data and Methods for Structure-Activity Relationship of Penem Antibiotic Sidechains for Use Against Mycobacteria Reveal Highly Active Compounds

### 2.1 General Methods and Instrumentation

All reagents and starting materials were purchased and used without further purification unless otherwise indicated. Anhydrous solvents were dried using an LC Technology Solutions (Salisbury, MA) SPBT-1 solvent purification system. Silica gel chromatography was performed using Sorbtech Silica Gel (60 Å, 40-75 mm particle size) or RediSep Rf disposable flash columns (60 Å, 40-63 μm irregular particle size) on a Teledyne ISCO (Lincoln, NE) CombiFlash EZ Prep. Preparative HPLC was carried out on the same instrument outfitted with a Phenomenex (Torrance, CA) Luna 10p C18(2) 100 Å column (250×21.20 mm ID). All ¹H- and ¹³C-NMR spectra were recorded on a Bruker (Billerica, MA) UltraShield 400 MHz or 300 MHz Avance spectrometer. The Johns Hopkins Chemistry Department Mass Spectrometry Facility determined exact masses by high resolution ultra-performance liquid chromatography-electrospray ionization mass spectrometry (UPLC-ESIMS) using a Waters (Milford, MA) Acquity/Xevo-G2.

### 2.2 General Procedure for Allyl Carbamate Protection of Nitrogen:

A round bottom flask was charged with the amino alcohol (1 equiv.) and a 1:1 mixture of water:tetrahydrofuran (THF) (1 M). The mixture was cooled to 0° C. and allyl chloroformate (1.2 equiv.) was added dropwise while stirring. Upon completion of addition, the pH of the mixture was adjusted to 10 with 4N aqueous NaOH. The reaction was allowed to proceed for 1 h. Aqueous brine solution was then added to the mixture and the organics were extracted three times with ethyl acetate (EtOAc). Organics were dried with anhydrous sodium sulfate and removed in vacuo. Product was then purified from the organic mixture using silica gel flash chromatography with an EtOAc/Hexanes mobile phase.

### 2.3 General Procedure for Mesylation of an Alcohol

Round bottom flask was charged with allyl carbamate-protected amino alcohol (1 equiv.). The oil was dissolved in dichloromethane (DCM) (0.2 M) and cooled to 0° C. Methanesulfonyl chloride (1.2 equiv.) was then added to the flask dropwise while stirring, followed by the dropwise addition of triethylamine (2 equiv.). The reaction was allowed to proceed for 1 h or until completion was indicated by thin layer chromatography. The reaction was quenched with water and the organics were extracted with three times with DCM. The organics were dried with anhydrous sodium sulfate and the solvent was removed in vacuo. The resulting oil was then purified using silica gel flash chromatography with an EtOAc/Hexanes mobile phase.

### 2.4 General Procedure for Installation of a Thioester:

The methanesulfonate (1 equiv.) added to a round bottom flask and dissolved in a 1:1 mixture of EtOAc to dimethylformamide (0.5 M). Potassium thioacetate (1.3 equiv.) was added to the solution in one portion. The flask was fitted with a reflux condenser and mixture was then stirred at 60° C. for 16 h. The mixture was poured into EtOAc and the salts were extracted with an aqueous brine solution. The aqueous layer was then back extracted three times with EtOAc. The organics were combined, dried with anhydrous sodium sulfate, and solvent was removed in vacuo. The resulting oil was then purified using silica gel flash chromatography with an EtOAc/Hexanes mobile phase.

### 2.5 General Procedure for Methyl Ester Formation:

The carboxylic acid (1 equiv.) was added to a round bottom flask and dissolved in methanol (0.6 M). A drop of conc. H₂SO₄ was added to the mixture. The flask was then fit with a reflux condenser and heated to 60° C. for 16 h. The reaction was allowed to cool to room temperature before the pH was adjust to 5 using 5% aqueous NaOH solution. The mixture was poured into EtOAc and the organic layer was washed with an aqueous brine solution. Organic layer was then dried with anhydrous sodium sulfate and concentrated in vacuo to afford the product as an oil.

### 2.6 General Procedure for Methyl Ester Reduction:

The methyl ester (1 equiv.) was dissolved in methanol (0.4 M) and cooled to 0° C. To the mixture NaBH₄ (2 equiv.) was added portion-wise. The reaction was stirred for 4 h before being quenched with 5% H₂SO₄ solution. EtOAc was added to the mixture and the organic layer was separated, dried with anhydrous sodium sulfate, and concentrated in vacuo. The oil was then purified used silica gel flash chromatography with an EtOAc/Hexanes mobile phase.

2.7 General Procedure for Sidechain Thiol Deprotection and β-Addition-Elimination into Penem Core:

The thioester protected side chain (1.5 equiv.) was dissolved in MeOH (0.4 M) and cooled to 0° C. While stirring, a sodium methoxide 5.4 M solution (9 equiv.) was added dropwise. The reaction was allowed to proceed for 2 h before being quenched with a dropwise addition of 6 N HCl until a pH of 4 was reached. The mixture was diluted with degassed EtOAc and washed with brine solution. The resulting organic layer was concentrated in vacuo. The resulting oil was dissolved in CH₃CN (0.3 M) and cooled to 0° C. before adding diisopropylethylamine (3 equiv.) dropwise to the flask. The mixture was added dropwise to a stirring mixture of the penem core 5 (1 equiv.) in CH₃CN (0.2 M) at 0° C. and allowed to react for 3 h. The mixture was then diluted into EtOAc and washed with brine. The aqueous layer was back extracted three times with EtOAc and the resulting organics were dried with anhydrous sodium sulfate. The solvent was removed in vacuo and the resulting oil was then purified using silica gel flash chromatography with an EtOAc/Hexanes mobile phase.

### 2.8 General Procedure for a One-Pot Allyl Carbamate and Allyl Ester Deprotection:

The penem protected with an allyl ester and an allyl carbamate (1 equiv.), barbituric acid (1.2 equiv.), and sodium benzenesulfinate (1.2 equiv.) were dissolved in THE (0.05 M). The mixture was degassed by bubbling of nitrogen gas through the mixture for 15 min. Palladium-tetrakis(triphenylphosphine) (0.1 equiv.) was then added to the mixture under nitrogen atmosphere and the reaction was allowed to proceed for 3 h. The solvent was then removed from the reaction in vacuo, and the resulting oil was partitioned into EtOAc and water. The organic layer was back extracted twice with water and the aqueous layers were combined. The mixture was then purified by HPLC using a C18 stationary phase and water/ACN+0.1% TFA mobile phase. The product fraction was then lyophilized to yield the powder form of the final penem antibiotic.

### 2.9 General Procedure for Reductive Amination:

The deprotected penem bearing a secondary amine (1 equiv.) was dissolved in a 3:1 water containing 0.1% formic acid to isopropyl alcohol (0.3 M). Either acetyl aldehyde (3 equiv.) or 4-oxotetrahydropyran (3 equiv.) was added to the mixture at 0° C. and let equilibrate for 5 min. NaBH(OAc)₃ (3 equiv.) was then added in one portion and the reaction was allowed to proceed for 16 h. Reaction progress was checked by UPLC-HRMS and upon completion, the product was purified using HPLC using a C18 stationary phase and water/ACN+0.1% TFA mobile phase. The product fraction was then lyophilized to yield the powder form of the final penem antibiotic.

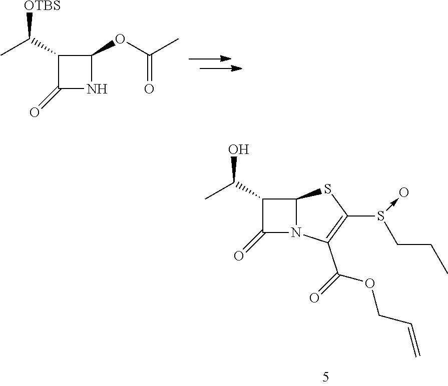 [SMILES: CC(=O)O[C@H]1NC(=O)[C@@H]1[C@@H](C)O[Si](C)(C)C(C)(C)C ; C=CCOC(=O)C1=C(S(=O)CCC)S[C@@H]2[C@@H]([C@@H](C)O)C(=O)N12]

### Allyl (5S,6R)-6-((R)-1-hydroxyethyl)-7-oxo-3-(propylsulfinyl)-4-thia-1-azabicyclo[3.2.0]hept-2-ene-2-carboxylate (5)

Multistep synthesis of compound 5 was performed as previously described. Batchelder et al., 2020.

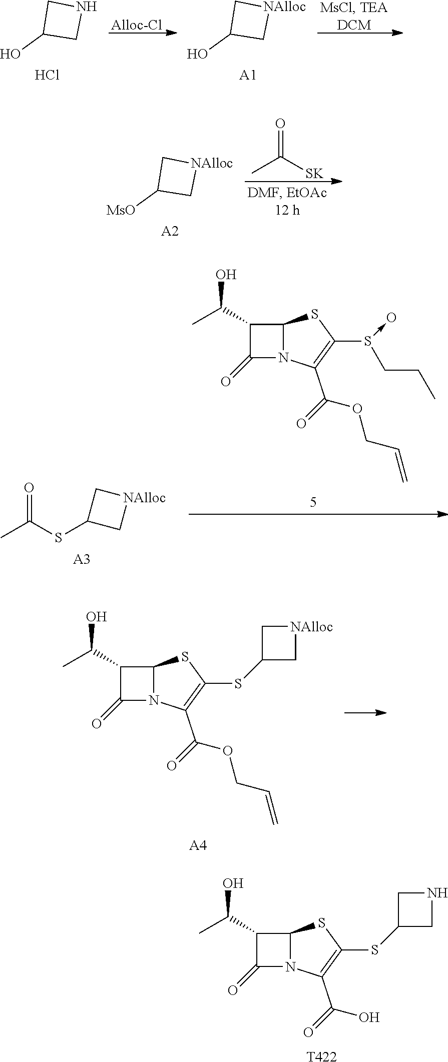

### Allyl 3-hydroxyazetidine-1-carboxylate (A1)

General procedure for allyl carbamate formation of an amino alcohol was used to convert 3-hydroxyazetidine hydrochloride to A1 (2.5 g, 96%). A1: ¹H NMR (400 MHz, CDCl₃) δ=5.91 (ddt, J=5.6, 10.4, 17.2 Hz, 1H), 5.29 (dq, J=1.6, 17.2 Hz, 1H), 5.21 (dq, J=1.3, 10.4 Hz, 1H), 4.63 (dddd, J=4.4, 6.6, 10.9, 10.9 Hz, 1H), 4.55 (ddd, J=1.4, 1.5, 5.6 Hz, 2H), 4.23 (ddd, J=1.0, 6.7, 9.7 Hz, 2H), 3.88 (ddd, J=1.2, 4.4, 9.5 Hz, 2H). ¹³C NMR (101 MHz, CDCl₃) δ=156.6, 132.6, 117.7, 65.8, 61.4, 59.0. HRMS (UPLC/MS), C₇H₁₁NO₃ [M+H] calculated: 158.0812; found: 158.0822.

### Allyl 3-methylsulfonyloxyazetidine-1-carboxylate (A2)

General procedure for mesylation of an alcohol was performed to convert A1 to A2 (3.3 g, 86%). A2: ¹H NMR (300 MHz, CDCl₃) δ=5.91 (ddt, J=5.7, 10.4, 17.2 Hz, 1H), 5.27 (m, 4H), 4.57 (dt, J=1.5, 5.6 Hz, 2H), 4.36 (ddd, J=1.1, 6.6, 10.3 Hz, 2H), 4.18 (ddd, J=1.2, 4.2, 10.6 Hz, 2H), 3.07 (s, 3H). ¹³C NMR (101 MHz, CDCl₃) δ=156.0, 132.5, 117.8, 67.5, 65.9, 56.6, 38.2. HRMS (UPLC/MS), C₈H₁₃NO₅S [M+H] calculated: 236.0587; found: 236.0592.

### Allyl 3-acetylthioazetidine-1-carboxylate (A3)

General procedure for installation of a thioester was used to convert A2 to A3 (1.8 g, 65%). A3: ¹H NMR (400 MHz, CDCl₃) δ=5.90 (ddt, J=5.2, 11.0, 17.1, 1H), 5.23 (ddd, J=0.5, 1.52, 17.2 Hz, 1H), 5.21 (dd, J=0.8, 10.4 Hz, 1H), 4.55 (d, J=5.6 Hz, 2H), 4.44 (t, J=8.7 Hz, 2H), 4.21 (tt, J=5.5, 8.1 Hz, 1H), 3.89 (dd, J=5.6, 9.4, 2H), 2.34 (s, 3H). ¹³C NMR (101 MHz, CDCl₃) δ=194.2, 155.6, 132.7, 117.4, 65.4, 55.9, 30.8, 30.2. HRMS (UPLC/MS), C₉H₁₃NO₃S [M+H] calculated: 216.0689; found: 216.0683.

### Allyl (5R,6S)-3-((1-((allyloxy)carbonyl)azetidin-3-yl)thio)-6-((R)-1-hydroxyethyl)-7-oxo-4-thia-1-azabicyclo[3.2.0]hept-2-ene-2-carboxylate (A4)

General procedure for sidechain thiol deprotection and 1-addition-elimination into the penem core was used to incorporate sidechain A3 into the penem core, A4 (815 mg, 61%). A4: ¹H NMR (400 MHz, CDCl₃) δ=5.93 (m, 2H), 5.68 (d, J=2.0 Hz, 1H), 5.42 (dq, J=1.6, 22.9 Hz, 1H), 5.24 (m, 1H), 5.26 (m, 2H), 4.73 (dddABq, J=0.6, 2.2, 7.4, 17.9 Hz, 2H), 4.56 (dt, J=2.1, 7.5 Hz, 2H), 4.40 (ddd, J=9.9, 12.0, 22.4 Hz, 2H), 4.24 (quin, J=8.7 Hz, 1H), 4.05 (m, 4H), 3.73 (dd, J=2.0, 9.1 Hz, 1H), 1.30 (d, J=8.4 Hz, 3H). ¹³C NMR (101 MHz, CDCl₃) δ=172.2, 159.6, 155.8, 152.0, 132.5, 131.6, 118.6, 118.0, 117.8, 71.5, 66.0, 65.8, 65.5, 65.1, 35.3, 21.8. HRMS (UPLC/MS), C₁₈H₂₂N₂O₆S₂ [M+H] calculated: 427.0992; found: 427.1000.

### (5R,6S)-3-(azctidin-3-ylthio)-6-((R)-1-hydroxyethyl)-7-oxo-4-thia-1-azabicyclo[3.2.0]hept-2-ene-2-carboxylic acid (T422)

The general procedure for a one-pot allyl carbamate and allyl ester deprotection was used to convert A4 to T422 (97 mg, 51%). T422: ¹H NMR (400 MHz, D₂O) δ=5.75 (s, 1H), 4.56 (m, 4H), 4.22 (m, 1H), 4.15 (m, 3H), 3.98 (d, J=5.5 Hz, 1H), 1.26 (d, J=6.3 Hz, 3H). ¹³C NMR (101 MHz, D₂O) δ=175.8, 162.7, 117.7, 114.8, 69.9, 64.9, 64.4, 52.2, 52.0, 37.2, 19.9. HRMS (UPLC/MS), C₁₁H₁₄N₂O₄S₂ [M+H] calculated: 303.0468; found: 303.0478.

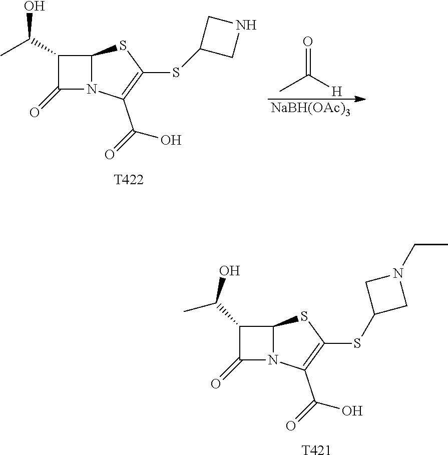

### (5R,6S)-3-((1-ethylazetidin-3-yl)thio)-6-((R)-1-hydroxyethyl)-7-oxo-4-thia-1-azabicyclo[3.2.0]hept-2-ene-2-carboxylic acid (T421)

General procedure for reductive amination was used with acetaldehyde to convert T422 to T421 (4 mg, 18%). T421: ¹H NMR (400 MHz, D₂O) δ=5.78 (s, 1H), 4.50 (m, 3H), 4.29 (m, 11H), 4.23 (t, J=6.0 Hz, 11H), 4.12 (m, 2H), 4.01 (dd, J=1.2, 5.6 Hz, 11H), 3.30 (m, 2H), 1.27 (d, J=6.4 Hz, 3H), 1.16 (dt, J=2.2, 7.2 Hz, 3H). HRMS (UPLC/MS), C₁₃H₁₈N₂O₄S₂[M+H] calculated: 331.0781; found: 331.0778.

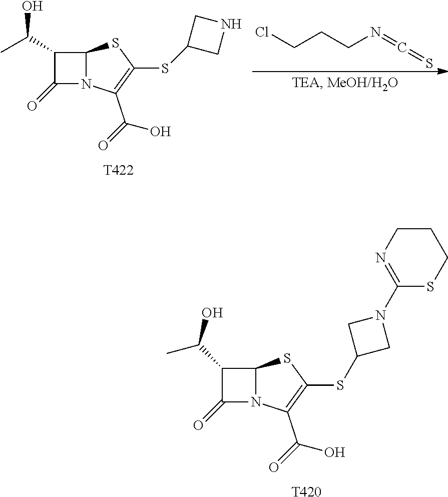

### (5R,6S)-3-((1-(5,6-dihydro-4H-1,3-thiazin-2-yl)azetidin-3-yl)thio)-6-((R)-1-hydroxyethyl)-7-oxo-4-thia-1-azabicyclo[3.2.0]hept-2-ene-2-carboxylic acid (T420)

The deprotected penem T422 (100 mg, 0.33 mmol) was dissolved in a methanol (220 L) and water (166 μL) mixture and cooled to 0° C. To the solution 3-chloropropyl isothiocyanate (34 μL, 0.33 mmol) was added followed by triethylamine (92 μL, 0.66 mmol). The reaction was allowed to proceed for 16 h. The mixture was then purified by HPLC using a C18 stationary phase and water/ACN+0.1% TFA mobile phase. The product fraction was then lyophilized to yield the powder form of the final penem antibiotic T420 (41 mg, 32%). T420: ¹H NMR (400 MHz, D₂O) S=5.72 (d, J=1.3 Hz, 1H), 4.59 (m, 2H), 4.37 (m, 1H), 4.12 (m, 3H), 3.94 (dd, J=1.3, 5.6 Hz, 1H), 3.38 (t, J=5.6 Hz, 2H), 3.15 (t, J=5.8 Hz, 2H), 2.05 (m, 2H), 1.21 (d, J=6.4 Hz, 3H). ¹³C NMR (101 MHz, D₂O) δ=175.9, 163.7, 162.4, 154.8, 117.8, 117.2, 114.9, 70.1, 65.1, 64.5, 40.8, 35.2, 26.1, 20.2, 20.0. HRMS (UPLC/MS), C₁₅H₁₉N₃O₄S₃[M+H] calculated: 402.0610; found: 402.0607.

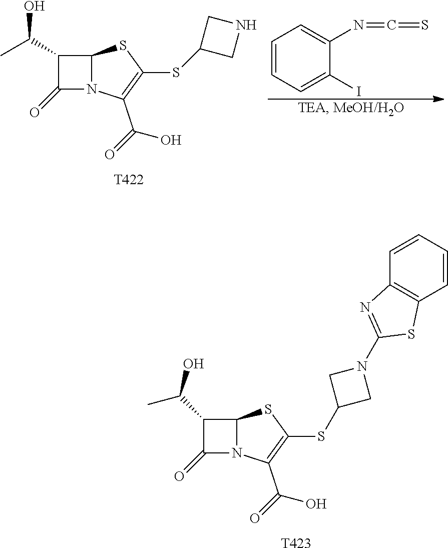

### (5R,6S)-3-((1-(benzo[d]thiazol-2-yl)azetidin-3-yl)thio)-6-((R)-1-hydroxyethyl)-7-oxo-4-thia-1-azabicyclo[3.2.0]hept-2-ene-2-carboxylic acid (T423)

The deprotected penem T422 (15 mg, 50 μmol) was dissolved in a methanol (12 μL) and water (12 μL) mixture and cooled to 0° C. To the solution 2-iodophenyl isothiocyanate (14 mg, 55 gmol) was added followed by the addition of triethylamine (14 μL, 100 gmol). The reaction was allowed to proceed for 16 h. The mixture was then purified by HPLC using a C18 stationary phase and water/ACN+0.1% TFA mobile phase. The product fraction was then lyophilized to yield the powder form of the final penem antibiotic T423 (4 mg, 19%). T423: ¹H NMR (400 MHz, DMSO-d6) δ=7.80 (d, J=7.5 Hz, 111), 7.51 (d, J=8.5 Hz, 1H), 7.30 (t, J=8.0 Hz, 11H), 7.10 (t, J=6.9 Hz, 11H), 6.56 (s, 11H), 5.69 (m, 11H), 5.19 (d, J=4.6 Hz, 111), 4.55 (m, 2H), 4.03 (m, 2H), 3.97 (m, 1H), 3.50 (m, 111), 1.15 (d, J=6.2 Hz, 3H). HRMS (UPLC/MS), C₁₈H₁₇N₃O₄S₃[M+H] calculated: 436.0454; found: 436.0453.

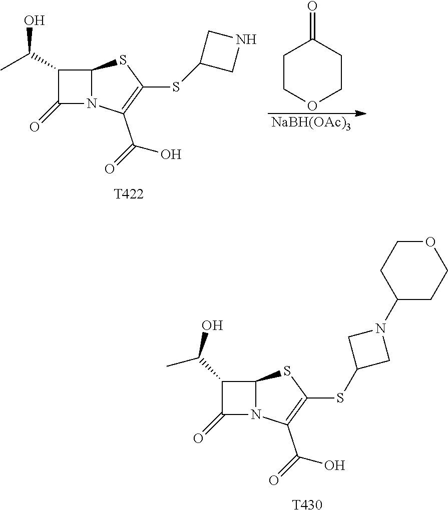

### (5R,6S)-6-((R)-1-hydroxyethyl)-7-oxo-3-((1-(tetrahydro-2H-pyran-4-yl)azetidin-3-yl)thio)-4-thia-1-azabicyclo[3.2.0]hept-2-ene-2-carboxylic acid (T430)

General procedure for reductive amination was used with 4-oxotetrahydropyran as the ketone to convert T422 to T430 (13 mg, 59%). T430:

¹H NMR (400 MHz, D₂O) δ=5.78 (d, J=1.4 Hz, 1H), 4.70 (m, 1H), 4.62 (m, 1H), 4.52 (quin., J=8.0 Hz, 1H), 4.38 (quin. J=3.8 Hz, 1H), 4.22 (m, 2H), 4.05 (d, J=8.6 Hz, 2H), 4.01 (d, J=5.6 Hz, 1H), 3.55 (m, 1H), 3.43 (t, J=11.2 Hz, 2H), 1.98 (m, 2H), 1.47 (dq, J=4.5, 11.9 Hz, 2H), 1.26 (d, J=6.5 Hz, 3H). HRMS (UPLC/MS), C₁₆H₂₂N₂O₅S₂ [M+H] calculated: 387.1043; found: 387.1047.

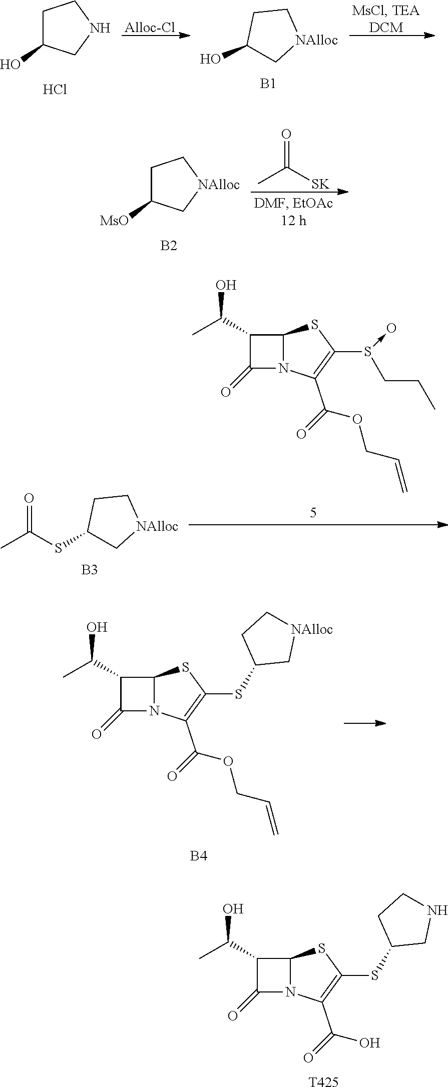

### Allyl (S)-3-hydroxypyrrolidine-1-carboxylate (B1)

General procedure for allyl carbamate formation of an amino alcohol was used to convert (S)-3-hydroxypyrrolidine hydrochloride to B1 (1.9 g, 80%). B1 (mixture of conformational isomers): ¹H NMR (400 MHz, CDCl₃) δ=5.94 (ddt, J=5.5, 10.4, 17.2 Hz, 1H), 5.30 (dq, J=1.6, 17.2 Hz, 1H), 5.30 (s, 1H), 5.20 (dq, J=1.4, 10.4 Hz, 1H), 4.59 (dt, J=1.5, 5.5 Hz, 2H), 4.48 (m, 1H), 3.54 (m, 3H), 3.44 (s, 0.7H), 3.41 (s, 0.3H), 2.00 (m, 2H), 1.86 (s, 1H). ¹³C NMR (101 MHz, CDCl₃) δ=155.0, 154.9, 132.9, 117.1, 70.6, 69.7, 67.0, 65.9, 65.6, 54.4, 53.9, 44.0, 43.7, 41.2, 33.8, 33.3. HRMS (UPLC/MS), C₈H₁₄NO₃ [M+H] calculated: 172.0968; found: 172.0975.

### Allyl (S)-3-((methylsulfonyl)oxy)pyrrolidine-1-carboxylate (B2)

General procedure for mesylation of an alcohol was performed to convert B1 to B2 (2.8 g, 52%). B2 (mixture of conformational isomers): ¹H NMR (400 MHz, CDCl₃) δ=5.80 (br s, 1H), 5.12 (m, 3H), 4.45 (br s, 2H), 3.49 (m, 4H), 2.92 (s, 3H), 2.13 (br s, 2H), 4.56 (d, J=6.6 Hz, 2H), 3.96 (m, 1H), 3.58 (m, 1H), 3.46 (m, 1H), 3.31 (m, 1H), 2.30 (s, 3H), 1.88 (m, 111). ¹³C NMR (101 MHz, CDCl₃) δ 153.8, 153.7, 132.4, 116.6, 79.7, 79.1, 65.1, 51.7, 51.4, 43.2, 42.8, 37.8, 31.8, 30.9. HRMS (UPLC/MS), C₉H₁₅NO₅S [M+H] calculated: 250.0744; found: 250.0757.

### Allyl (R)-3-(acetylthio)pyrrolidine-1-carboxylate (B3)

General procedure for installation of a thioester was used to convert B2 to B3 (1.6 g, 63%). B3: (mixture of conformational isomers) ¹H NMR (400 MHz, CDCl₃) δ=5.91 (m, 1H), 5.27 (d, J=22.9 Hz, 1H), 5.17 (d, J=13.9, 1H), 4.56 (d, J=6.6 Hz, 2H), 3.96 (m, 1H), 3.81 (m, 1H), 3.46 (m, 1H), 3.31 (m, 1H), 2.30 (s, 3H), 1.88 (m, 1H). ¹³C NMR (101 MHz, CDCl₃) δ=195.1, 195.0, 154.4, 133.0, 117.3, 117.2, 65.7, 51.6, 51.4, 45.0, 44.6, 41.1, 40.5, 32.0, 30.9, 30.6. HRMS (UPLC/MS), C₁₀H₁₅NO₃S [M+H] calculated: 230.0845; found: 230.0849.

### Allyl (5R,6,S)-3-(((R)-1-((allyloxy)carbonyl)pyrrolidin-3-yl)thio)-6-((R)-1-hydroxyethyl)-7-oxo-4-thia-1-azabicyclo[3.2.0]hept-2-ene-2-carboxylate (B4)

General procedure for sidechain thiol deprotection and j-addition-elimination into penem core 5 was used to incorporate sidechain B3 into the penem core, B4 (200 mg, 52%). B4 (mixture of rotational isomers): ¹H NMR (400 MHz, CDCl₃) δ=5.89 (m, 2H), 5.67 (d, J=1.4 Hz, 1H), 5.37 (dq, J=1.3, 17.2 Hz, 1H), 4.69 (dABq, J=5.4, 13.4 Hz, 2H), 4.54 (d, J=5.5 Hz, 2H), 4.17 (quin, J=6.4 Hz, 1H), 3.87 (m, 1H), 3.78 (m, 1H), 3.69 (m, 1H), 3.51 (m, 311), 2.32 (sept, J=6.8 Hz, 111), 1.98 (m, 111), 1.30 (d, J=6.3 Hz, 3H). ¹³C NMR (101 MHz, CDCl₃) δ=172.2, 159.7, 154.5, 154.4, 152.8, 152.8, 132.9, 132.8, 118.5, 118.0, 117.7, 117.6, 71.5, 71.4, 66.1, 65.8, 65.6, 64.7, 53.0, 52.6, 446.6, 45.9, 44.9, 44.5, 32.7, 32.0, 21.9. HRMS (UPLC/MS), C₁₉H₂₄N₂O₆S₂ [M+H] calculated: 441.1149; found: 441.1152.

### (5R,6S)-6-((R)-1-hydroxyethyl)-7-oxo-3-(((R)-pyrrolidin-3-yl)thio)-4-thia-1-azabicyclo[3.2.0]hept-2-ene-2-carboxylic acid (T425)

The general procedure for a one-pot allyl carbamate and allyl ester deprotection was used to convert B4 to T425 (21.4 mg, 30%). T425: ¹H NMR (400 MHz, D₂O) δ=5.75 (d, J=1.3 Hz, 1H), 4.21 (quin, J=6.3 Hz, 1H), 4.11 (tt, J=5.2, 7.2 Hz, 1H), 3.98 (dd, J=1.4, 5.7 Hz, 1H), 3.73 (dd, J=7.4, 12.9 Hz, 1H), 3.47 (m, 1H), 3.37 (m, 2H), 2.57 (sex, J=7.4 Hz, 1H), 2.14 (sex, J=6.2 Hz, 1H), 1.25 (d, J=6.4 Hz, 3H). ¹³C NMR (101 MHz, D₂O) δ=175.9, 162.5, 154.3, 118.1, 70.1, 64.6, 64.5, 51.1, 44.8, 44.7, 31.3, 20.0. HRMS (UPLC/MS), C₁₂H₁₆N₂O₄S₂[M+H] calculated: 317.0624; found: 317.0627.

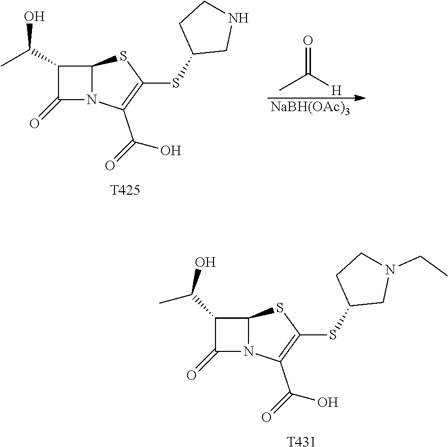

### (5R,6S)-3-(((R)-1-ethylpyrrolidin-3-yl)thio)-6-((R)-1-hydroxyethyl)-7-oxo-4-thia-1-azabicyclo[3.2.0]hept-2-ene-2-carboxylic acid (T431)

General procedure for reductive amination was used with acetaldehyde to convert T425 to T431 (8.5 mg, 44%). T431: ¹H NMR (400 MHz, D₂O) δ=5.69 (s, 1H), 4.15 (t, J=5.6 Hz, 1H), 3.93 (d, J=5.6 Hz, 1H), 3.64 (m, 3H), 3.20 (m, 5H), 2.51 (m, 1H), 1.96 (m, 1H), 1.20 (m, 6H). HRMS (UPLC/MS), C₁₉H₂₀N₄O₄S₂[M+H] calculated: 345.0937; found: 345.0939.

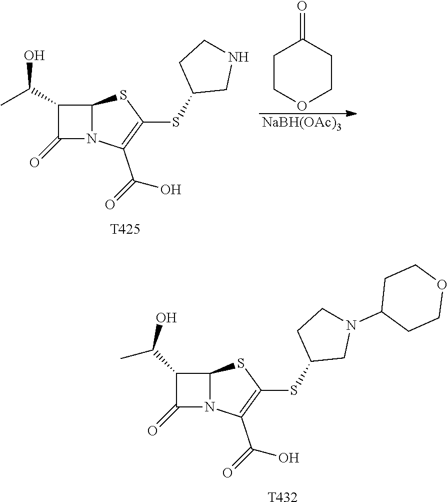

### (5R,6S)-6-((R)-1-hydroxyethyl)-7-oxo-3-(((R)-1-(tetrahydro-2H-pyran-4-yl)pyrrolidin-3-yl)thio)-4-thia-1-azabicyclo[3.2.0]hept-2-ene-2-carboxylic acid (T432)

General procedure for reductive amination was used with 4-oxotetrahydropyran as the ketone to convert T425 to T432 (31 mg, 41%). T432: ¹H NMR (400 MHz, D₂O) δ=5.78 (d, J=1.04 Hz, 1H), 4.24 (t, J=5.96 Hz, 1H), 4.06 (br s, J=9.92, 4H), 3.72 (m, 3H), 3.46 (t, J=12.0 Hz, 4H), 3.27 (m, 1H), 2.73 (m, 1H), 2.08 (m, 4H), 1.73 (m, 2H), 1.23 (d, J=6.4 Hz, 3H). ¹³C NMR (101 MHz, DMSO-d6) δ=173.4, 160.9, 71.3, 65.2, 64.5, 63.9, 60.6, 60.5, 48.6, 43.0, 30.8, 28, 9, 21.5. HRMS (UPLC/MS), C₁₇H₂₄N₂O₅S₂[M+H] calculated: 401.1199; found: 401.1197.

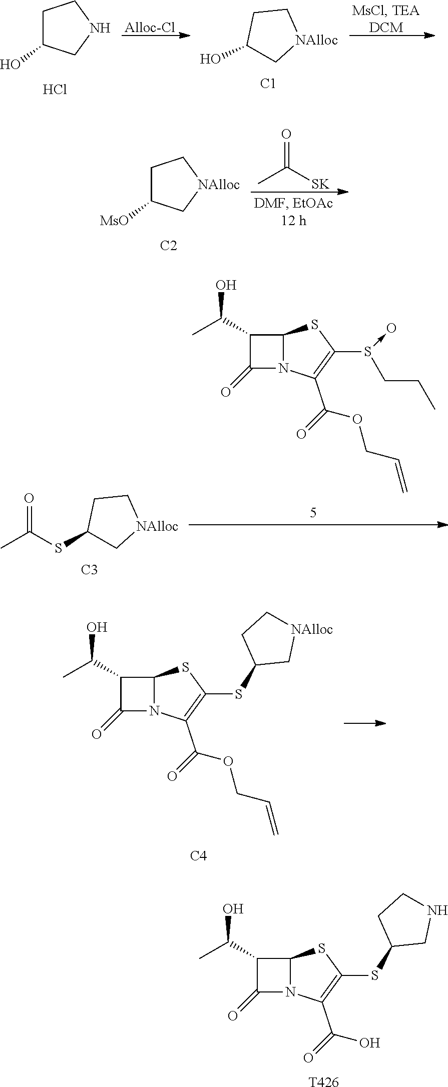

### Allyl (R)-3-hydroxypyrrolidine-1-carboxylate (C1)

General procedure for allyl carbamate formation of an amino alcohol was used to convert (R)-3-Hydroxypyrrolidine hydrochloride to C1 (3.5 g, 85%). C1 (mixture of conformational isomers): 1H NMR (400 MHz, CDCl₃) δ=5.87 (tdd, J=4.7, 11.4, 17 Hz, 1H), 5.24 (d, J=17.2 Hz, 1H), 5.14 (d, J=10.4 Hz, 1H), 4.51 (d, J=5.4 Hz, 2H), 4.37 (br s, 1H), 3.46 (m, 2H), 3.41 (t, J=3.9 Hz, 1H), 3.35 (m, 1H), 1.89 (br s, 2H). ¹³C NMR (101 MHz, CDCl₃) δ=155.1, 155.0, 133.0, 133.0, 117.2, 70.7, 69.8, 65.8, 54.5, 54.0, 44.1, 43.8, 33.9, 33.4. HRMS (UPLC/MS), C₈H₁₄NO₃ [M+H] calculated: 172.0968; found: 172.0967.

### Allyl (R)-3-((methylsulfonyl)oxy)pyrrolidine-1-carboxylate (C₂)

General procedure for mesylation of an alcohol was performed to convert C₁ to C₂ (4.5 g, 87%). C₂ (mixture of conformational isomers): ¹H NMR (400 MHz, CDCl₃) δ=5.87 (tdd, J=5.3, 10.6, 16.6 Hz, 1H), 5.24, (m, 2H), 5.16 (d, J=10.3 Hz, 1H), 4.53 (d, J=4.7, 2H), 3.63 (m, 3H), 3.47 (m, 111), 2.99 (s, 3H), 2.23 (br s, 111), 2.11 (m, 111). ¹³C NMR (101 MHz, CDCl₃) δ=154.6, 132.7, 117.5, 79.6, 66.0, 52.1, 43.6, 38.6, 31.5. HRMS (UPLC/MS), C₉H₁₅NO₅S [M+H] calculated: 250.0744; found: 250.0752.

### Allyl (S)-3-(acetylthio)pyrrolidine-1-carboxylate (C3)

General procedure for installation of a thioester was used to convert C2 to C3 (1.65, 40%). C3 (mixture of conformational isomers): ¹H NMR (400 MHz, CDCl₃) δ=5.89 (tdd, J=4.6, 10.6, 17 Hz, 1H), 5.25 (d, J=17.2 Hz, 1H), 5.16 (d, J=10.4 Hz, 1H), 4.54 (d, J=4 Hz, 2H), 3.95 (quin, J=6.5 Hz, 2H), 3.78 (dt, J=6.5, 11.5 Hz, 111), 3.46 (m, 2H), 3.29 (m, 1H), 2.29 (s, 3H), 2.26 (m, 1H), 1.86 (oct, J=6.4 Hz, 1H). ¹³C NMR (101 MHz, CDCl₃) δ=195.2, 195.1, 154.5, 133.1, 133.0, 117.4, 117.3, 65.8, 51.7, 51.5, 45.1, 44.7, 41.2, 40.6, 32.1, 31.0, 30.7. HRMS (UPLC/MS), C₁₀H₁₅NO₃S [M+H][M+H] calculated: 230.0845; found: 230.0856.

### Allyl (5R,6S)-3-(((S)-1-((allyloxy)carbonyl)pyrrolidin-3-yl)thio)-6-((R)-1-hydroxyethyl)-7-oxo-4-thia-1-azabicyclo[3.2.0]hept-2-ene-2-carboxylate (C4)

General procedure for sidechain thiol deprotection and β-addition-elimination into penem core was used to incorporate sidechain C3 into the penem core, C4 (670 mg, 34%). C4: ¹H NMR (400 MHz, CDCl₃) δ=5.89 (m, 2H), 5.67 (d, J=1.4 Hz, 1H), 5.37 (dq, J=1.4, 17.2 Hz, 1H), 5.26 (dq, J=1.5, 17.3 Hz, 1H1), 5.19 (t, J=10.4 Hz, 2H), 4.74 (dtABq, J=1.4, 5.4, 13.5 Hz, 1H), 4.62 (dABq, J=5.8, 13.3 Hz, 1H), 4.54 (d, J=5.5 Hz, 2H), 4.17 (quin, J=5.9 Hz, 1H), 3.87 (dd, J=6.6, 11.6 Hz, 1H), 3.78 (m, 1H), 3.69 (m, 1H), 3.51 (m, 3H), 2.32 (quin, J=6.6 Hz, 1H), 1.98 (m, 1H), 1.30 (d, J=6.3 Hz, 3H). ¹³C NMR (101 MHz, CDCl₃) δ=172.3, 159.6, 154.5, 154.3, 152.9, 132.8, 131.7, 130.8, 118.4, 118.0, 117.7, 117.6, 71.4, 66.0, 65.7, 65.5, 65.0, 64.8, 52.9, 52.3, 46.6, 45.9, 44.9, 44.4, 32.6, 32.2, 21.8. HRMS (UPLC/MS), C₁₉H₂₄N₂O₆S₂[M+H] calculated: 441.1149; found: 441.1144. (5R,6S)-6-((R)-1-hydroxyethyl)-7-oxo-3-(((S)-pyrrolidin-3-yl)thio)-4-thia-1-azabicyclo[3.2.0]hept-2-ene-2-carboxylic acid (T426): The general procedure for a one-pot allyl carbamate and allyl ester deprotection was used to convert C₄ to T426 (97 mg, 51%). T426: ¹H NMR (400 MHz, D₂O) δ=5.67 (s, 1H), 4.13 (quin, J=6.0 Hz, 1H), 4.03 (m, 1H), 3.89 (d, J=5.7 Hz, 1H), 3.73 (dd, J=7.0, 12.8 Hz, 1H), 3.35 (m, 4H), 2.43 (sex, J=7.64 Hz, 1H₁), 1.98 (sex, J=7.2 Hz, 1H₁), 1.17 (d, J=6.4 Hz, 3H). ¹³C NMR (101 MHz, D₂O) S=175.8, 162.3, 154.5, 118.1, 70.0, 64.8, 64.5, 51.4, 44.9, 44.7, 30.9, 20.0. FIRMS (UPLC/MS), C₁₂H₁₆N₂O₄S₂[M+H] calculated: 317.0624; found: 317.0622.

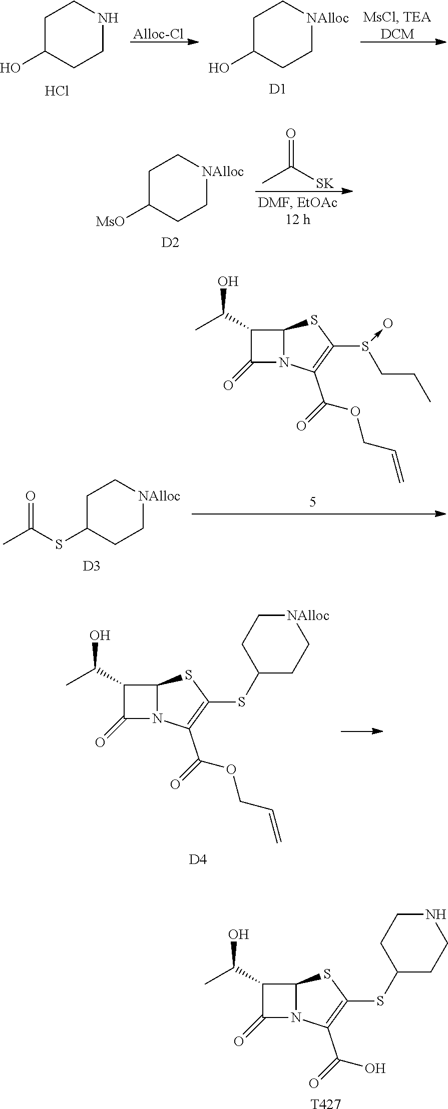

### Allyl 4-hydroxypiperidine-1-carboxylate (D1)

General procedure for allyl carbamate formation of an amino alcohol was used to convert 4-hydroxypiperidine hydrochloride to D1 (1.7 g, 93%). DL: ¹H NMR (400 MHz, CDCl₃) δ=5.94 (m, 1H), 5.29 (m, 1H), 5.21 (m, 1H), 4.59 (m, 2H), 3.89 (m, 3H), 3.13 (ddd, J=3.4, 9.5, 13.2 Hz, 2H), 1.87 (m, 2H), 1.65 (br s, 1H), 1.49 (m, 2H). ¹³C NMR (101 MHz, CDCl₃) δ=155.0, 133.1, 117.3, 65.8, 54.4, 43.9, 33.7. HRMS (UPLC/MS), C₉H₁₅NO₃ [M+H] calculated: 186.1125; found: 186.1146.

### Allyl 4-((methylsulfonyl)oxy)piperidine-1-carboxylate (D2)

General procedure for mesylation of an alcohol was performed to convert D1 to D2 (1.5 g, 63%). D2: ¹H NMR (400 MHz, CDCl₃) δ=5.66 (ddt, J=5.4, 10.3, 17.2 Hz, 11H), 5.01 (dd, J=1.6, 17.3 Hz, 1H), 4.93 (dd, J=1.4, 10.4 Hz, 1H), 4.60 (sept, J=3.6 Hz, 1H), 4.29 (d, J=5.4 Hz, 2H), 3.45 (m, 2H), 3.11 (m, 2H), 2.79 (s, 3H), 1.71 (m, 2H), 1.53 (m, 2H). ¹³C NMR (101 MHz, CDCl₃) δ=154.5, 133.0, 117.0, 77.3, 65.7, 40.4, 38.3, 31.3. HRMS (UPLC/MS), C₁₀H₁₇NO₅S [M+H] calculated: 264.0900; found: 264.0897.

### Allyl 4-(acetylthio)piperidine-1-carboxylate (D3)

General procedure for installation of a thioester was used to convert D2 to D3 (470 mg, 34%). D3: ¹H NMR (400 MHz, CDCl₃) δ=5.92 (ddt, J=5.5, 10.4, 17.2 Hz, 111), 5.29 (dq, J=1.6, 17.2 Hz, 1H), 5.20 (dq, J=1.3, 10.4 Hz, 1H), 4.57 (dt, J=1.4, 5.5 Hz, 2H), 3.91 (d, J=12.7 Hz, 2H), 3.62 (tt, J=4.0, 10.2 Hz, 1H), 3.13 (t, J=10.8 Hz, 2H), 2.31 (s, 3H), 1.92 (m, 2H), 1.56 (m, 2H). ¹³C NMR (101 MHz, CDCl₃) δ=195.0, 155.0, 133.1, 117.4, 66.1, 43.5, 39.9, 31.8, 30.8. HRMS (UPLC/MS), C₁₁H₁₇NO₃S [M+H] calculated: 244.1002; found: 244.0998.

### Allyl (5R,6S)-3-((1-((allyloxy)carbonyl)piperidin-4-yl)thio)-6-((R)-1-hydroxyethyl)-7-oxo-4-thia-1-azabicyclo[3.2.0]hept-2-ene-2-carboxylate (D4)

General procedure for sidechain thiol deprotection and β-addition-elimination into penem core was used to incorporate sidechain D3 into the penem core, D4 (185 mg, 47%). D4: ¹H NMR (400 MHz, CDCl₃) δ=5.9 (m, 2H), 5.63 (s, 1H), 5.38 (d, J=17.2 Hz, 1H), 5.26 (d, J=17.2 Hz, 1H), 5.19 (t, J=10.2 Hz, 2H), 4.68 (dABq, J=5.4, 13.4 Hz, 2H), 4.54 (d, J=5.4 Hz, 2H), 4.18 (t, J=6.2 Hz, 1H), 3.99 (d, J=12.8 Hz, 2H), 3.69 (d, J=7.1 Hz, 1H), 3.31 (m, 1H), 3.03 (br s, 3H), 2.07 (m, 2H), 1.62 (m, 2H), 1.31 (d, J=6.2 Hz, 3H). ¹³C NMR (101 MHz, CDCl₃) δ=172.1, 159.7, 155.0, 152.7, 132.9, 131.8, 118.4, 118.0, 117.6, 71.3, 66.3, 65.7, 65.5, 64.3, 46.3, 43.1, 43.1, 32.7, 32.7, 21.9. HRMS (UPLC/MS), C₂₀H₂₆N₂O₆S₂ [M+H] calculated: 455.1305; found: 455.1300.

### (5R,6S)-6-((R)-1-hydroxyethyl)-7-oxo-3-(piperidin-4-ylthio)-4-thia-1-azabicyclo[3.2.0]hept-2-ene-2-carboxylic acid (T427)

The general procedure for a one-pot allyl carbamate and allyl ester deprotection was used to convert D4 to T427 (37.7 mg, 52%). T427: ¹H NMR (400 MHz, D₂O) δ=5.76 (d, J=1.4, 1H), 4.25 (quin, J=6.0 Hz, 1H), 4.00 (dd, J=1.4, 5.8 Hz, 1H), 3.60 (tt, J=3.9, 10.2 Hz, 1H), 3.46 (m, 3H), 3.15 (m, 3H), 2.38 (m, 2H), 2.23 (m, 1H), 1.91 (m, 1H), 1.30 (d, J=6.4 Hz, 3H). ¹³C NMR (101 MHz, D₂O) δ=174.5, 161.0, 153.6, 113.6, 68.7, 63.3, 63.0, 41.6, 41.5, 27.8, 27.5, 18.8. HRMS (UPLC/MS), C₁₃H₁₈N₂O₄S₂ [M+H] calculated: 331.0781; found: 331.0792.

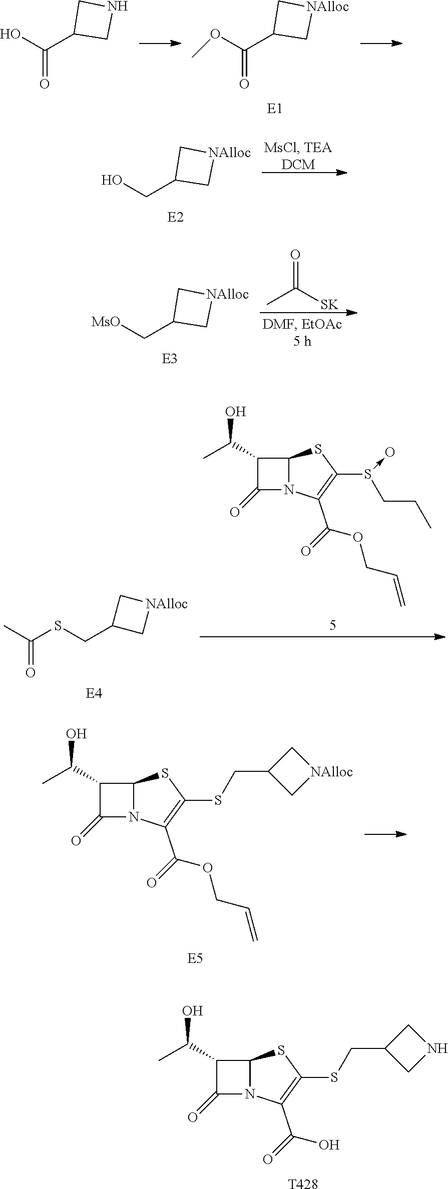

### 1-Allyl 3-methyl azetidine-1,3-dicarboxylate (E1)

The general procedure for allyl carbamate protection of nitrogen was first performed azetidine-3-carboxylic acid followed by the general procedure for methyl ester formation to give E1 (1.8 g, 93%). E1 (Mixture of conformational isomers): ¹H NMR (400 MHz, CDCl₃) δ=5.74 (m, 1H), 5.12 (dt, J=1.6, 17.2 Hz, 1H), 5.03 (d, J=10.4, 1H), 4.37 (m, 2H), 4.01 (m, 4H), 3.58 (m, 3H), 3.26 (quin, J=7.9 Hz, 1H). ¹³C NMR (101 MHz, CDCl₃) δ=172.2, 155.6, 132.4, 117.0, 65.1, 51.8, 51.3, 31.9. HRMS (UPLC/MS), C₉H₁NO₄ [M+H] calculated: 200.0917; found: 200.0918.

### Allyl 3-(hydroxymethyl)azetidine-1-carboxylate (E2)

The general procedure for methyl ester reduction was used to convert E1 to E2 (1.1 g, 70%). E2: ¹H NMR (400 MHz, CDCl₃) δ=5.82 (ddt, J=5.5, 11.5, 17.2 Hz, 1H), 5.20 (d, J=17.2 Hz, 1H), 5.12 (d, J=10.4 Hz, 1H), 4.45 (d, J=5.4 Hz, 2H), 3.96 (t, J=8.4 Hz, 2H), 3.66 (m, 5H), 2.67 (m, 1H). ¹³C NMR (101 MHz, CDCl₃) δ=156.5, 132.7, 117.4, 65.5, 63.8, 51.6, 51.1, 30.8. HRMS (UPLC/MS), C₈H₁₃O₃[M+H] calculated: 172.0968; found: 172.0966.

### Allyl 3-(((methylsulfonyl)oxy)methyl)azetidine-1-carboxylate (E3)

The general procedure for mesylation of an alcohol was used to convert E2 to E3 (1.6 g, 98%). E3: ¹H NMR (400 MHz, CDCl₃) δ=5.72 (ddt, J=4.5, 10.5, 17.2 Hz, 1H), 5.11 (dq, J=1.6, 17.2 Hz, 1H), 5.02 (dq, J=1.3, 10.4 Hz, 1H), 4.34 (dt, J=1.4, 5.5 Hz, 2H), 4.17 (d, J=6.5 Hz, 2H), 3.92 (m, 2H), 3.61 (m, 2H) 2.87 (s, 3H), 2.81 (m, 1H). ¹³C NMR (101 MHz, CDCl₃) δ=155.7, 132.5, 116.9, 70.0, 65.0, 50.7, 36.8, 27.9. HRMS (UPLC/MS), C₉H₁₅NO₅S [M+H] calculated: 250.0744; found: 250.0765.

### Allyl 3-((acetylthio)methyl)azetidine-1-carboxylate (E4)

The general procedure for installation of a thioester was used to convert E3 to E4 (923 mg, 66%). E4: ¹H NMR (400 MHz, CDCl₃) δ=5.75 (ddt, J=5.5, 10.5, 17.2 Hz, 1H), 5.13 (dd, J=1.6, 17.2 Hz, 1H), 5.03 (dd, J=1.4, 10.4 Hz, 1H), 4.37 (dt, J=1.4, 5.5 Hz, 2H), 3.91 (t, J=8.5 Hz, 2H), 3.49 (dd, J=5.5, 8.7 Hz, 2H), 2.96 (d, J=7.5 Hz, 2H), 2.65 (m, 1H), 2.19 (s, 3H). ¹³C NMR (101 MHz, CDCl₃) δ=194.6, 155.8, 132.7, 117.1, 65.2, 53.5, 32.2, 30.3, 28.7. HRMS (UPLC/MS), C₁₀H₁₅NO₃S [M+H] calculated: 230.0845; found: 230.0846.

### Allyl (5R,6,S)-3-(((1-((allyloxy)carbonyl)azetidin-3-yl)methyl)thio)-6-((R)-1-hydroxyethyl)-7-oxo-4-thia-1-azabicyclo[3.2.0]hept-2-ene-2-carboxylate (E5)

The general procedure for sidechain thiol deprotection and β-addition-elimination into penem core 5 was used to incorporate sidechain E4 into the penem core to generate E5 (245 mg, 48%). E5: ¹H NMR (400 MHz, CDCl₃) δ=5.91 (m, 2H), 5.65 (br s, 1H), 5.39 (d, J=17.2 Hz, 1H), 5.27 (d, J=17.2 Hz, 1H), 5.21 (m, 2H), 4.70 (ddABq, J=1.1, 5.4, 13.5 Hz, 2H), 4.53 (dd, J=1.0, 5.5 Hz, 2H), 4.20 (quin, J=6.2 Hz, 1H), 4.12 (t, J=8.6 Hz, 2H), 3.72 (m, 3H), 3.18 (dABq, J=8.0, 12.8 Hz, 2H), 2.87 (m, 1H), 1.33 (d, J=6.3 Hz, 3H). ¹³C NMR (101 MHz, CDCl₃) δ=172.1, 159.7, 156.3, 153.6, 132.8, 131.8, 118.5, 117.8, 71.4, 65.8, 65.8, 65.6, 64.6, 53.9, 39.6, 29.3, 22.0. HRMS (UPLC/MS), C₁₉H₂₄N₂O₆S₂[M+H] calculated: 441.1149; found: 441.1157.

### (5R,6S)-3-((azetidin-3-ylmethyl)thio)-6-((R)-1-hydroxyethyl)-7-oxo-4-thia-1-azabicyclo[3.2.0]hept-2-ene-2-carboxylic acid (T428)

The general procedure for a one-pot allyl carbamate and allyl ester deprotection was used to convert E5 to T428 (74.1 mg, 89%). T428: ¹H NMR (400 MHz, D₂O) δ=5.75 (s, 1H), 4.26 (t, J=6.1 Hz, 1H), 3.99 (d, J=11.5 Hz, 2H), 2.99 (m, 2H), 2.11 (d, J=13.8 Hz, 2H), 2.03 (m, 1H), 1.48 (m, 2H), 1.31 (d, J=6.2 Hz, 3H). ¹³C NMR (101 MHz, D₂O) δ=175.9, 162.5, 117.7, 114.8, 69.8, 64.6, 43.7, 40.7, 34.0, 27.4, 20.1. HRMS (UPLC/MS), C₁₂H₁₆N₂O₄S₂ [M+H] calculated: 317.0624; found: 317.0626.

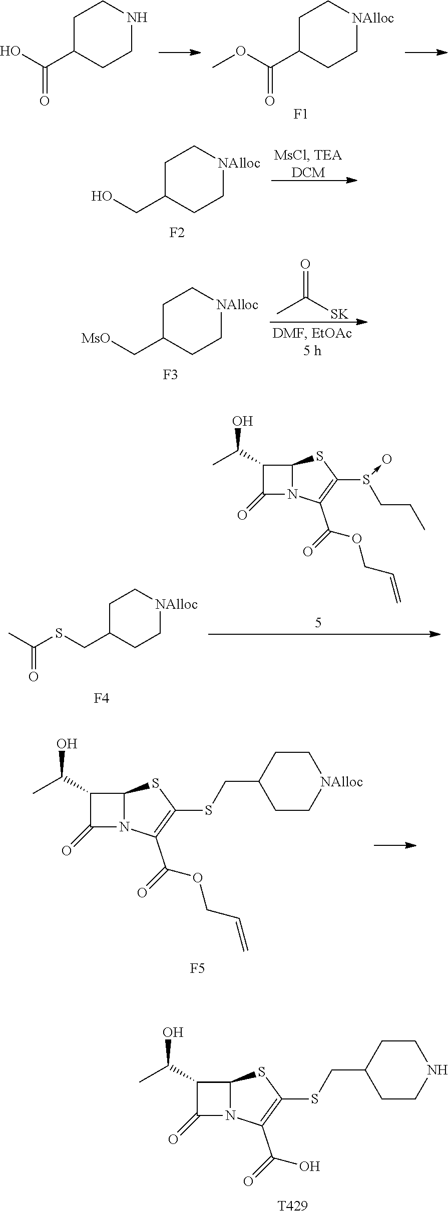

### 1-Allyl 4-methyl piperidine-1,4-dicarboxylate (F1)

The general procedure for allyl carbamate protection of nitrogen was first performed on piperidine-4-carboxylic acid followed by the general procedure for methyl ester formation to give F1 (1.7 g, 96%). F1: ¹H NMR (400 MHz, CDCl₃) δ=5.93 (ddt, J=5.8, 10.4, 17.2 Hz, 1H), 5.29 (tt, J=1.6, 17.2 Hz, 1H), 5.20 (tt, J=1.4, 10.4 Hz, 1H), 4.58 (td, J=1.4, 5.5 Hz, 2H), 4.09 (m, 2H), 3.69 (s, 3H), 2.92 (br s, 2H), 2.48 (tt, J=3.9, 11.0 Hz, 1H), 1.91 (br s, 1H), 1.89 (br s, 1H), 1.65 (m, 3H). ¹³C NMR (101 MHz, CDCl₃) δ=174.8, 155.0, 133.0, 117.3, 66.0, 51.8, 43.1, 40.8, 27.8. HRMS (UPLC/MS), C₁₁H₁₇NO₄ [M+H] calculated: 228.1230; found: 228.1229.

### Allyl 4-(hydroxymethyl)piperidine-1-carboxylate (F2)

The general procedure for methyl ester reduction was used to convert F1 to F2 (1 g, 70%). F2: ¹H NMR (400 MHz, CDCl₃) δ=5.77 (ddt, J=5.5, 10.5, 17.2 Hz, 1H), 5.13 (dq, J=1.5, 17.2 Hz, 1H), 5.05 (dq, J=1.3, 10.5 Hz, 1H), 4.41 (d, J=5.4 Hz, 2H), 4.01 (br d, J=12.2 Hz, 2H), 3.54 (br s, 1H), 3.28 (d, J=6.3 Hz, 2H), 2.62 (br s, 2H), 1.59 (br d, J=13.2 Hz, 2H), 1.50 (m, 1H), 0.99 (dq, J=4.4, 8.2 Hz, 2H). ¹³C NMR (101 MHz, CDCl₃) δ=155.0, 132.8, 117.0, 66.7, 65.7, 43.6, 38.4, 28.4. HRMS (UPLC/MS), C₁₀H₁₇NO₃ [M+H] calculated: 200.1281; found: 200.1283.

### Allyl 4-(((methylsulfonyl)oxy)methyl)piperidine-1-carboxylate (F3)

The general procedure for mesylation of an alcohol was used to convert F2 to F3 (783 mg, 86%). F3: ¹H NMR (400 MHz, CDCl₃) δ=5.80 (ddt, J=5.5, 10.2, 17.2 Hz, 1H), 5.16 (dq, J=1.6, 17.2 Hz, 1H), 5.07 (dq, J=1.2, 10.5 Hz, 1H), 4.46 (d, J=5.5 Hz, 2H), 4.07 (br d, J=9.3 Hz, 2H), 3.94 (d, J=6.4 Hz, 2H), 2.89 (s, 3H), 2.67 (br s, 2H), 1.83 (m, 1H), 1.64 (br d, J=12.9 Hz, 2H), 1.11 (dq, J=4.5, 12.6 Hz, 2H). ¹³C NMR (101 MHz, CDCl₃) δ=154.7, 132.9, 117.0, 73.2, 65.6, 43.1, 36.9, 35.5, 27.8. HRMS (UPLC/MS), C₁₁H₁₉NO₅S [M+H] calculated: 278.1057; found: 278.1067.

### Allyl 4-((acetylthio)methyl)piperidine-1-carboxylate (F4)

The general procedure for installation of a thioester was used to convert F3 to F4 (448 mg, 64%). F4: ¹H NMR (400 MHz, CDCl₃) δ=5.82 (ddt, J=5.5, 10.4, 17.2 Hz, 1H), 5.17 (dd, J=1.6, 17.2 Hz, 1H), 5.08 (dd, J=1.4, 10.4 Hz, 1H), 4.46 (d, J=5.5 Hz, 2H), 4.05 (br s, 2H), 2.72 (d, J=6.7 Hz, 2H), 2.63 (br s, 2H), 2.22 (s, 3H), 1.65 (br d, J=12.9 Hz, 2H), 1.53 (m, 1H), 1.06 (dq, J=4.4, 12.6 Hz, 2H). ¹³C NMR (101 MHz, CDCl₃) δ=195.1, 154.8, 133.1, 117.0, 65.7, 43.6, 36.2, 34.7, 31.0, 30.4. HRMS (UPLC/MS), C₁₂H₁₉NO₃S [M+H] calculated: 258.1158; found: 258.1163.

### Allyl (5R,6S)-3-(((1-((allyloxy)carbonyl)piperidin-4-yl)methyl)thio)-6-((R)-1-hydroxyethyl)-7-oxo-4-thia-1-azabicyclo[3.2.0]hept-2-ene-2-carboxylate (F5)

The general procedure for sidechain thiol deprotection and β-addition-elimination into penem core was used to incorporate sidechain F4 into the penem core to generate F5 (210 mg, 39%). F5: ¹H NMR (400 MHz, CDCl₃) δ=5.93 (m, 2H), 5.63 (d, J=1.4 Hz, 1H), 5.40 (dd, J=1.4, 17.2 Hz, 1H), 5.28 (dd, J=1.5, 17.2 Hz, 1H), 5.23 (dd, J=1.5, 10.5 Hz, 1H), 5.19 (dd, J=1.4, 10.6 Hz, 1H), 4.71 (dABq, J=5.4, 13.5 Hz, 2H), 4.56 (d, J=5.5 Hz, 2H), 4.19 (m, 1H), 3.70 (dd, J=1.0, 6.7 Hz, 1H), 2.83 (m, 4H), 2.39 (br s, 1H), 1.86 (m, 2H), 1.76 (m, 2H), 1.34 (d, J=6.3 Hz, 3H), 1.17 (m, 2H). ¹³C NMR (101 MHz, CDCl₃) δ=172.1, 159.9, 155.5, 155.2, 133.2, 132.0, 118.4, 117.5, 71.2, 66.2, 65.7, 64.4, 43.9, 42.1, 37.1, 31.3, 22.1. HRMS (UPLC/MS), C₂₁H₂₈N₂O₆S₂ [M+H] calculated: 469.1462; found: 469.1476.

### (5R,6S)-6-((R)-1-hydroxyethyl)-7-oxo-3-((piperidin-4-ylmethyl)thio)-4-thia-1-azabicyclo[3.2.0]hept-2-ene-2-carboxylic acid (T429)

The general procedure for a one-pot allyl carbamate and allyl ester deprotection was used to convert F5 to T429 (53.4 mg, 74%). T429: ¹H NMR (400 MHz, D₂O) δ=5.75 (s, 111), 4.26 (t, J=6.2 Hz, 1H), 3.99 (d, J=5.6 Hz, 111), 3.46 (m, 3H), 3.00 (m, 4H), 2.06 (m, 4H), 1.48 (m, 4H), 1.48 (m, 3H), 1.31 (d, J=6.2 Hz, 3H). ¹³C NMR (101 MHz, D₂O) δ=175.9, 162.5, 117.7, 114.8, 69.8, 64.5, 64.2, 43.7, 40.7, 34.0, 27.4, 27.3, 20.1. HRMS (UPLC/MS), C₁₄H₂₀N₂O₄S₂ [M+H] calculated: 345.0937; found: 345.0940.

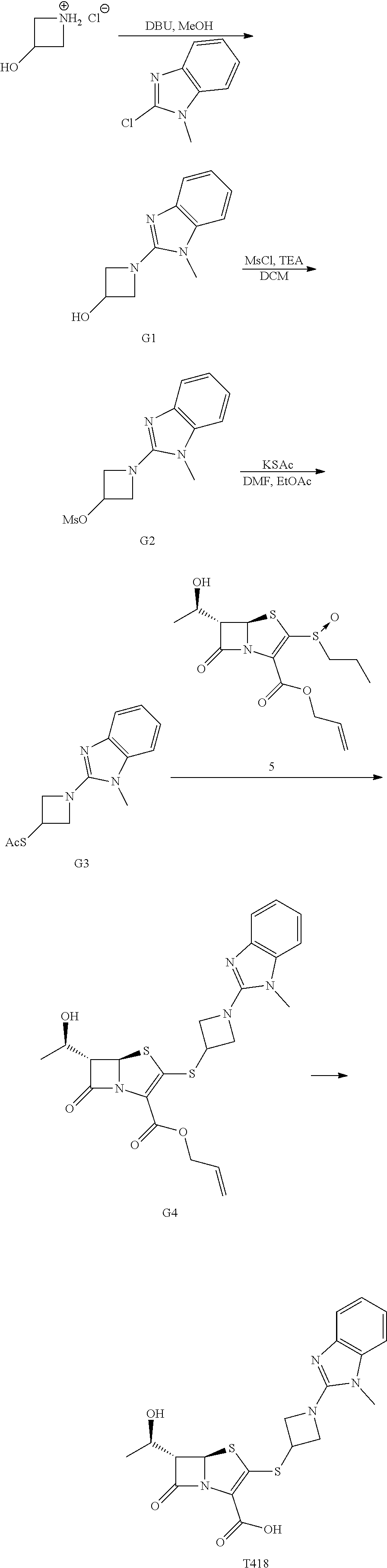

### 1-(1-Methyl-1H-benzo[d]imidazol-2-yl)azetidin-3-ol (G1)

A flask was charged with 3-hydroxyazetidine hydrochloride (492 mg, 4.5 mmol), 1-N-methyl-2-chlorobenzimidazole (reagent prepare as previously described, Bharautam et al., 2016) (679 mg, 4.1 mmol), and 3 Å molecular sieves. The mixture was then suspended in methanol (12 mL) and stirred under nitrogen for 10 min. To the mixture, 1,8-diazabicyclo[5.4.0]undec-7-ene (1.3 mL, 8.5 mmol) was added dropwise and the mixture was heated to 65° C. for 16 h. The reaction mixture was then filtered, and the retentate was washed with methanol. The filtrate was then concentrated, suspended in EtOAc, and extracted with water. The aqueous layer was then washed twice with EtOAc, the organic layers were combined, dried with anhydrous sodium sulfate, and concentrated in vacuo. The mixture was then purified using silica gel flash chromatography with isopropanol/dichloromethane mobile phase to yield G1 (629 mg, 76%). G1: ¹H NMR (400 MHz, CDCl₃) δ=7.46 (d, J=7.5 Hz, 1H), 7.08 (m, 3H), 4.80 (tt, J=6.4, 5.0 Hz, 1H), 4.44 (dd, J=8.0, 7.0 Hz, 2H), 4.15 (dd, J=5.0, 8.7 Hz, 2H), 3.40 (s, 3H). ¹³C NMR (101 MHz, CDCl₃) δ=157.21, 141.21, 135.44, 121.55, 120.27, 116.53, 107.60, 62.72, 62.57, 29.56. HRMS (UPLC/MS), C₁₁H₁₃N₃O [M+H] calculated: 204.1137; found: 204.1137.

### 1-(1-Methyl-1H-benzo[d]imidazol-2-yl)azetidin-3-yl methanesulfonate (G2)

General procedure for mesylation of an alcohol was performed to convert G1 to G2 (720 mg, 43%). G2: ¹H NMR (400 MHz, CDCl₃) δ=7.52 (d, J=6.8 Hz, 1H), 7.14 (m, 3H), 5.38 (tt, J=4.6, 6.5 Hz, 1H), 4.55 (dd, J=6.8, 9.8 Hz, 2H), 4.39 (dd, J=4.6, 9.9 Hz, 2H), 3.51 (s, 3H), 3.07 (s, 3H). ¹³C NMR (101 MHz, CDCl₃) δ=156.37, 141.35, 135.57, 121.71, 120.70, 117.27, 107.82, 68.31, 59.51, 38.34, 29.53. HRMS (UPLC/MS), C₁₃H₁₅N₃O₃S [M+H] calculated: 282.0907; found: 282.0947.

### S-(1-(1-Methyl-1H-benzo[d]imidazol-2-yl)azetidin-3-yl) ethanethioate (G3)

The general procedure for installation of a thioester was used to convert G2 to G3 (546 mg, 82%). G3: ¹H NMR (400 MHz, CDCl₃) δ=7.49 (d, J=7.4 Hz, 1H), 7.09 (m, 3H), 4.62 (t, J=8.1 Hz, 2H), 4.39 (m, 1H), 4.12 (dd, J=5.9, 8.2 Hz, 2H), 3.45 (s, 3H), 2.32 (s, 3H). ¹³C NMR (101 MHz, CDCl₃) δ=194.65, 156.36, 141.11, 135.29, 121.51, 120.38, 116.88, 107.57, 59.13, 32.20, 30.26, 29.43. HRMS (UPLC/MS), C₁₃H₁₅N₃OS [M+H] calculated: 262.1009; found: 262.1050.

### Allyl (5R,6S)-6-((R)-1-hydroxyethyl)-3-((1-(1-methyl-1H-benzo[d]imidazol-2-yl)azetidin-3-yl)thio)-7-oxo-4-thia-1-azabicyclo[3.2.0]hept-2-ene-2-carboxylate (G4)

General procedure for sidechain thiol deprotection and β-addition-elimination into penem core 5 was used to add side chain G3 into penem core to give G4 (51.8 mg, 13%). G4: ¹H NMR (400 MHz, CDCl₃) δ=7.54 (d, J=7.4 Hz, 1H), 7.18 (m, 3H), 5.96 (tdd, J=5.5, 10.6, 17.1 Hz, 1H), 5.78 (s, 1H), 5.43 (dd, J=1.2, 17.2 Hz, 1H), 5.26 (dd, J=0.9, 10.4 Hz, 1H), 4.77 (m, 5H), 4.28 (m, 4H), 3.77 (d, J=7.24, 1H), 3.58 (s, 3H), 1.38 (d, J=6.1 Hz, 3H). ¹³C NMR (101 MHz, CDCl₃) δ=172.3, 159.6, 152.0, 131.7, 122.6, 122.5, 121.6, 121.5, 118.6, 117.8, 116.2, 116.1, 108.2, 71.7, 65.8, 65.6, 60.3, 59.6, 37.0, 29.9, 22.0, 14.2. HRMS (UPLC/MS), C₂₂H₂₄N₄O₄S₂ [M+H] calculated: 473.1312; found: 473.1320.

### (5R,6S)-6-((R)-1-hydroxyethyl)-3-((1-(1-methyl-1H-benzo[d]imidazol-2-yl)azetidin-3-yl)thio)-7-oxo-4-thia-1-azabicyclo[3.2.0]hept-2-ene-2-carboxylic acid (T418)

The allyl ester-protected penem G4 (46.6 mg, 99 μmol) and sodium benzenesulfinate (21.1 mg, 129 μmol) was dissolved in THF (2 ml). The mixture was degassed via bubbling of nitrogen gas through the mixture for 15 min. Palladium-tetrakis(triphenylphosphine) (10 mg, 9 μmol) was then added to the mixture under nitrogen atmosphere and the reaction was allowed to proceed for 3 h. The solvent was then removed in vacuo, and the resulting oil was partitioned into EtOAc and water. The organic layer was back extracted twice with water and the aqueous layers were combined. The mixture was then purified by HPLC using a C18 stationary phase and water/ACN+0.1% TFA mobile phase. The product fraction was then lyophilized to yield the powder form of the final penem antibiotic T418 (19 mg, 44%). ¹H NMR (400 MHz, D₂O) δ=7.38 (m, 1H), 7.30 (m, 1H), 7.26 (m, 2H), 5.64 (d, J=1.3 Hz, 1H), 4.92 (m, 2H), 4.44 (m, 2H), 4.00 (dq, J=6.4, 6.4 Hz, 1H), 3.67 (dd, J=1.3, 6.8 Hz, 1H), 3.63 (s, 3H), 3.19 (quin, J=1.6 Hz, 1H), 1.17 (d, J=6.3 Hz, 3H). ¹³C NMR (101 MHz, D₂O) δ=174.6, 159.8, 151.2, 133.4, 130.5, 125.8, 125.3, 112.8, 11.4, 72.9, 66.7, 66.4, 61.9, 61.5, 38.0, 30.6, 21.9. HRMS (UPLC/MS), C₁₉H₂₀N₄O₄S₂ [M+H] calculated: 433.0999; found: 433.1015.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Tuberculosis https://www.who.int/health-topics/tuberculosis #tab=tab_1 (accessed Jul. 26, 2021).
Migliori, G. B.; Tiberi, S.; Zumla, A.; Petersen, E.; Chakaya, J. M.; Wejse, C.; Torrico, M. M.; Duarte, R.; Alffenaar, J. W.; Schaaf, H. S.; Marais, B. J.; Cirillo, D. M.; Alagna, R.; Rendon, A.; Pontali, E.; Piubello, A.; Figueroa, J.; Ferlazzo, G.; Garcia-Basteiro, A.; Centis, R.; Visca, D.; D'Ambrosio, L.; Sotgiu, G.; Abu Arkub, T.; Akkerman, O. W.; Aleksa, A.; Belilovski, E.; Bernal, E.; Blanc, F. X.; Boeree, M.; Borisov, S.; Bruchfeld, J.; Cadiianos Loidi, J.; Caminero, J. A.; Carvalho, A. C.; Cebrian Gallardo, J. J.; Charalampos, M.; Danila, E.; Davies Forsman, L.; Denholm, J.; Dheda, K.; Diel, R.; Diktanas, S.; Dobler, C.; Enwerem, M.; Esposito, S.; Escobar Salinas, N.; Filippov, A.; Formenti, B.; Garcia Garcia, J. M.; Goletti, D.; Gomez Rosso, R.; Gualano, G.; Isaakidis, P.; Kaluzhenina, A.; Koirala, S.; Kuksa, L.; Kunst, H.; Li, Y.; Magis-Escurra, C.; Manfrin, V.; Manga, S.; Manika, K.; Marchese, V.; Martinez Robles, E.; Maryandyshev, A.; Matteelli, A.; Mariani, A.; Mazza-Stalder, J.; Mello, F.; Mendoza, L.; Mesi, A.; Miliauskas, S.; Mustafa Hamdan, H.; Ndjeka, N.; Nieto Marcos, M.; Ottenhoff, T. H. M.; Palmero, D. J.; Palmieri, F.; Papavasileiou, A.; Payen, M. C.; Pontarelli, A.; Pretti Dalcolmo, M.; Quirós Fernandez, S.; Romero, R.; Rossato Silva, D.; Santos, A. P.; Seaworth, B.; Sinitsyn, M.; Skrahina, A.; Solovic, I.; Spanevello, A.; Tadolini, M.; Torres, C.; Udwadia, Z.; van den Boom, M.; Volchenkov, G. V.; Yedilbayev, A.; Zaleskis, R.; Zellweger, J. P. MDR/XDR-TB Management of Patients and Contacts: Challenges Facing the New Decade. The 2020 Clinical Update by the Global Tuberculosis Network. Int. J. Infect. Dis. 2020, 92, S15-S25.
Diel, R.; Ringshausen, F.; Richter, E.; Welker, L.; Schmitz, J.; Nienhaus, A. Microbiological and Clinical Outcomes of Treating Non-Mycobacterium Avium Complex Nontuberculous Mycobacterial Pulmonary Disease: A Systematic Review and Meta-Analysis. Chest 2017, 152 (1), 120-142.
Jarand, J.; Levin, A.; Zhang, L.; Huitt, G.; Mitchell, J. D.; Daley, C. L. Clinical and Microbiologic Outcomes in Patients Receiving Treatment for Mycobacterium Abscessus Pulmonary Disease. Clin. Infect. Dis. 2011, 52 (5), 565-571.
Daley, C. L.; Iaccarino, J. M.; Lange, C.; Cambau, E.; Wallace, R. J.; Andrejak, C.; Böttger, E. C.; Brozek, J.; Griffith, D. E., Guglielmetti, L.; Huitt, G. A.; Knight, S. L.; Leitman, P.; Marras, T. K.; Olivier, K. N.; Santin, M.; Stout, J. E.; Tortoli, E.; Van Ingen, J.; Wagner, D.; Winthrop, K. L. Treatment of Nontuberculous Mycobacterial Pulmonary Disease: An Official ATS/ERS/ESCMID/IDSA Clinical Practice Guideline. Clin. Infect. Dis. 2020, 71 (4), 905-913.
Hamad, B. The Antibiotics Market. Nat. Rev. Drug Discov. 2010, 9 (9), 675-676.
Robinson, H. J. TOXICITY AND EFFICACY OF PENICILLIN. J Pharmacol. Exp. Ther. 1943, 77 (1), 70.

Smith, M. I.; Emmart, E. W. The Action of Penicillium Extracts in Experimental Tuberculosis. Public Heal. Reports 1944, 59 (13), 417.
Story-Roller, E.; Lamichhane, G. Have We Realized the Full Potential of β-Lactams for Treating Drug-Resistant TB?IUBMB Life 2018, 70 (9), 881.
Dhar, N.; Dubeé, V.; Ballell, L.; Cuinet, G.; Hugonnet, J. E.; Signorino-Gelo, F.; Barros, D.; Arthur, M.; McKinney, J. D. Rapid Cytolysis of Mycobacterium Tuberculosis by Faropenem, an Orally Bioavailable β-Lactam Antibiotic. Antimicrob. Agents Chemother. 2015, 59 (2), 1308-1319.
Batchelder, H. R.; Story-Roller, E.; Lloyd, E. P.; Kaushik, A.; Bigelow, K. M.; Maggioncalda, E. C.; Nuermberger, E. L.; Lamichhane, G.; Townsend, C. A. Development of a Penem Antibiotic against Mycobacteroides Abscessus. Commun. Biol. 2020, 3 (1), 1-5.
Hugonnet, J. E.; Tremblay, L. W.; Boshoff, H. I.; Barry, C. E.; Blanchard, J. S. Meropenem-Clavulanate Is Effective against Extensively Drug-Resistant Mycobacterium Tuberculosis. Sciencee 2009, 323 (5918), 1215-1218.
Tiberi, S.; Payen, M. C.; Sotgiu, G.; D'Ambrosio, L.; Guizado, V. A.; Alffenaar, J. W.; Arbex, M. A.; Caminero, J. A.; Centis, R.; De Lorenzo, S.; Gaga, M.; Gualano, G.; Arias, A. J. R.; Scardigli, A.; Skrahina, A.; Solovic, I.; Sulis, G.; Tadolini, M.; Akkerman, O. W.; Arrascue, E. A.; Aleska, A.; Avchinko, V.; Bonini, E. H.; Marin, F. A. C.; López, L. C.; De Vries, G.; Dore, S.; Kunst, H.; Matteelli, A.; Moschos, C.; Palmieri, F.; Papavasileiou, A.; Spanevello, A.; Vasquez, D. V.; Viggiani, P.; White, V.; Zumla, A.; Migliori, G. B. Effectiveness and Safety of Meropenem/Clavulanate-Containing Regimens in the Treatment of MDR- and XDR-TB. Eur. Respir. J. 2016, 47 (4), 1235-1243.
Diacon, A. H.; van der Merwe, L.; Barnard, M.; von Groote-Bidlingmaier, F.; Lange, C.; Garcia-Basteiro, A. L.; Sevene, E.; Ballell, L.; Barros-Aguirre, D. β-Lactams against Tuberculosis—New Trick for an Old Dog?N. Engl. J. Med. 2016, 375 (4), 393-394.
De Jager, V.; Gupte, N.; Nunes, S.; Barnes, G. L.; van Wijk, R. C.; Mostert, J.; Dorman, S. E.; Abulfathi, A. A.; Upton, C. M.; Faraj, A.; Nuermberger, E. L.; Lamichhane, G.; Svensson, E. M.; Simonsson, U.; Diacon, A. H.; Dooley, K. E. Early Bactericidal Activity of Meropenem Plus Clavulanate (+/−Rifampin) For TB. The COMRADE Randomized, Phase 2 Trial. Am. J. Respir. Crit. Care Med 2022.
Iannazzo, L.; Soroka, D.; Triboulet, S.; Fonvielle, M.; Compain, F.; Dubée, V.; Mainardi, J. L.; Hugonnet, J. E.; Braud, E.; Arthur, M.; Etheve-Quelquejeu, M. Routes of Synthesis of Carbapenems for Optimizing Both the Inactivation of L,D-Transpeptidase Ldt_{Mt1} of Mycobacterium Tuberculosis and the Stability toward Hydrolysis by β-Lactamase BlaC. J. Med Chem. 2016, 59 (7), 3427-3438.
Saidjalolov, S.; Edoo, Z.; Fonvielle, M.; Mayer, L.; Iannazzo, L.; Arthur, M.; Etheve-Quelquejeu, M.; Braud, E. Synthesis of Carbapenems Containing Peptidoglycan Mimetics and Inhibition of the Cross-Linking Activity of a Transpeptidase of 1,D Specificity. Chem. —A Eur. J. 2021, 27 (10), 3542-3551.
Gupta, R.; Al-Kharji, N. M. S. A.; Alqurafi, M. A.; Nguyen, T. Q.; Chai, W.; Quan, P.; Malhotra, R.; Simcox, B. S.; Mortimer, P.; Brammer Basta, L. A.; Rohde, K. H.; Buynak, J. D. Atypically Modified Carbapenem Antibiotics Display Improved Antimycobacterial Activity in the Absence of β-Lactamase Inhibitors. ACS Infect. Dis. 2021, 7 (8), 2425-2436.
Kitano, K.; Tomasz, A. Triggering of Autolytic Cell Wall Degradation in Escherichia Coli by Beta-Lactam Antibiotics, Antimicrob. Agents Chemother. 1979, 16 (6), 838.
Lavollay, M.; Arthur, M.; Fourgeaud, M.; Dubost, L.; Marie, A.; Veziris, N.; Blanot, D.; Gutmann, L.; Mainardi, J. L. The Peptidoglycan of Stationary-Phase Mycobacterium Tuberculosis Predominantly Contains Cross-Links Generated by L,D-Transpeptidation. J. Bacteriol. 2008, 190 (12), 4360-4366.
Gupta, R.; Lavollay, M.; Mainardi, J. L.; Arthur, M.; Bishai, W. R.; Lamichhane, G. The Mycobacterium Tuberculosis Protein Ldt Mt2 Is a Nonclassical Transpeptidase Required for Virulence and Resistance to Amoxicillin. Nat. Med. 2010, 16 (4), 466-469.
Blumberg, P. M.; Strominger, J. L. Interaction of Penicillin with the Bacterial Cell: Penicillin-Binding Proteins and Penicillin-Sensitive Enzymes. Bacteriol. Rev. 1974, 38 (3), 291-335.
Lavollay, M.; Fourgeaud, M.; Herrmann, J. L.; Dubost, L.; Marie, A.; Gutmann, L.; Arthur, M.; Mainardi, J. L. The Peptidoglycan of Mycobacterium Abscessus Is Predominantly Cross-Linked by L,D-Transpeptidases. J. Bacteriol. 2011, 193 (3), 778-782.
Erdemli, S. B.; Gupta, R.; Bishai, W. R.; Lamichhane, G.; Amzel, L. M.; Bianchet, M. A. Targeting the Cell Wall of Mycobacterium Tuberculosis: Structure and Mechanism of L,D-Transpeptidase 2. Structure 2012, 20 (12), 2103-2115.
Kim, H. S.; Kim, J.; Im, H. N.; Yoon, J. Y.; An, D. R.; Yoon, H. J.; Kim, J. Y.; Min, H. K.; Kim, S. J.; Lee, J. Y.; Han, B. W.; Suh, S. W. Structural Basis for the Inhibition of Mycobacterium Tuberculosis L,D-Transpeptidase by Meropenem, a Drug Effective against Extensively Drug-Resistant Strains. Acta Crystallogr. Sect. D Biol. Crystallogr. 2013, 69 (3), 420-431.
Dubée, V.; Triboulet, S.; Mainardi, J. L.; Etheve-Quelquejeu, M.; Gutmann, L.; Marie, A.; Dubost, L.; Hugonnet, J. E.; Arthur, M. Inactivation of Mycobacterium Tuberculosis L,D-Transpeptidase Ldt_{Mt1} by Carbapenems and Cephalosporins. Antimicrob. Agents Chemother. 2012, 56 (8), 4189-4195.
Cordillot, M.; Dubée, V.; Triboulet, S.; Dubost, L.; Marie, A.; Hugonnet, J. E.; Arthur, M.; Mainardia, J. L. In Vitro Cross-Linking of Mycobacterium Tuberculosis Peptidoglycan by L,D-Transpeptidases and Inactivation of These Enzymes by Carbapenems. Antimicrob. Agents Chemother. 2013, 57 (12), 5940-5945.
Kumar, P.; Kaushik, A.; Lloyd, E. P.; Li, S.-G.; Mattoo, R.; Ammerman, N. C.; Bell, D. T.; Perryman, A. L.; Zandi, T. A.; Ekins, S.; Ginell, S. L.; Townsend, C. A.; Freundlich, J. S.; Lamichhane, G. Non-Classical Transpeptidases Yield Insight into New Antibacterials. Nat. Chem. Biol. 2017a, 13, 54-61.
Steiner, E. M.; Schneider, G.; Schnell, R. Binding and Processing of 3-Lactam Antibiotics by the Transpeptidase Ldt_{Mt2} from Mycobacterium Tuberculosis. FEBS J. 2017, 284 (5), 725-741.
Kumar, P.; Chauhan, V.; Silva, J. R. A.; Lameira, J.; D'Andrea, F. B.; Li, S. G.; Ginell, S. L.; Freundlich, J. S.; Alves, C. N.; Bailey, S.; Cohen, K. A.; Lamichhane, G. Mycobacterium Abscessus L,D-Transpeptidases Are Susceptible to Inactivation by Carbapenems and Cephalosporins but Not Penicillins. Antimicrob. Agents Chemother. 2017b, 61 (10).
Bianchet, M. A.; Pan, Y. H.; Basta, L. A. B.; Saavedra, H.; Lloyd, E. P.; Kumar, P.; Mattoo, R.; Townsend, C. A.; Lamichhane, G. Structural Insight into the Inactivation of Mycobacterium Tuberculosis Non-Classical Transpeptidase Ldt_{Mt2} by Biapenem and Tebipenem. BMC Biochem. 2017, 18 (1).
Wang, F.; Cassidy, C.; Sacchettini, J. C. Crystal Structure and Activity Studies of the Mycobacterium Tuberculosis β-Lactamase Reveal Its Critical Role in Resistance to β-Lactam Antibiotics. Antimicrob. Agents Chemother. 2006, 50 (8), 2762-2771.
Kumar, G.; Galanis, C.; Batchelder, H. R.; Townsend, C. A.; Lamichhane, G. Penicillin Binding Proteins and β-Lactamases of Mycobacterium Tuberculosis: Reexamination of the Historical Paradigm. mSphere 2022, 7(1) e00039-22.
Tremblay, L. W.; Fan, F.; Blanchard, J. S. Biochemical and Structural Characterization of Mycobacterium Tuberculosis β-Lactamase with the Carbapenems Ertapenem and Doripenem. Biochemistry 2010, 49 (17), 3766-3773.
Lohans, C. T.; Chan, H. T. H.; Malla, T. R.; Kumar, K.; Kamps, J. J. A. G.; McArdle, D. J. B.; van Groesen, E.; de Munnik, M.; Tooke, C. L.; Spencer, J.; Paton, R. S.; Brem, J.; Schofield, C. J. Non-Hydrolytic β-Lactam Antibiotic Fragmentation by L,D-Transpeptidases and Serine β-Lactamase Cysteine Variants. Angew. Chemie—Int. Ed. 2019, 58 (7), 1990-1994.
Zandi, T. A.; Townsend, C. A. Competing Off-Loading Mechanisms of Meropenem from an L,D-Transpeptidase Reduce Antibiotic Effectiveness. Proc. Natl. Acad. Sci. U.S.A 2021, 118 (27).
Kumar, P.; Arora, K.; Lloyd, J. R.; Lee, I. Y.; Nair, V.; Fischer, E.; Boshoff, H. I. M.; Barry, C. E. Meropenem Inhibits D,D-Carboxypeptidase Activity in Mycobacterium Tuberculosis. Mol. Microbiol. 2012, 86 (2), 367-381.
Walkup, G. K.; You, Z.; Ross, P. L.; Allen, E. K. H.; Daryaee, F.; Hale, M. R.; O'Donnell, J.; Ehmann, D. E.; Schuck, V. J. A.; Buurman, E. T.; Choy, A. L.; Hajec, L.; Murphy-Benenato, K.; Marone, V.; Patey, S. A.; Grosser, L. A.; Johnstone, M.; Walker, S. G.; Tonge, P. J.; Fisher, S. L. Translating Slow-Binding Inhibition Kinetics into Cellular and in Vivo Effects. Nat. Chem. Biol. 2015, 11 (6), 416-423.
Copeland, R. A.; Pompliano, D. L.; Meek, T. D. Drug-Target Residence Time and Its Implications for Lead Optimization. Nat. Rev. Drug Discov. 2006, 5 (9), 730-739.
Lu, H.; Tonge, P. J. Drug-Target Residence Time: Critical Information for Lead Optimization. Curr. Opin. Chem. Biol. 2010, 14 (4), 467-474.
Kaushik, A.; Ammerman, N. C.; Tasneen, R.; Story-Roller, E.; Dooley, K. E.; Dorman, S. E.; Nuermberger, E. L.; Lamichhane, G. In Vitro and in Vivo Activity of Biapenem against Drug-Susceptible and Rifampicin-Resistant Mycobacterium Tuberculosis. J. Antimicrob. Chemother. 2017, 72 (8), 2320-2325.
Kaushik, A.; Ammerman, N. C.; Lee, J.; Martins, O.; Kreiswirth, B. N.; Lamichhane, G.; Parrish, N. M.; Nuermberger, E. L. In Vitro Activity of the New β-Lactamase Inhibitors Relebactam and Vaborbactam in Combination with β-Lactams against Mycobacterium Abscessus Complex Clinical Isolates. Antimicrob. Agents Chemother. 2019, 63 (3), e02623-18.
CLSI. in Performance standards for susceptibility testing of Mycobacteria, Nocardia spp., and other Aerobic Actinomycetes Vol. Supplement M62 (Ed: Woods, GL) (Clinical and Laboratory Standards Institute, 2018).
Basta, L. A. B.; Ghosh, A.; Pan, Y.; Jakoncic, J.; Lloyd, E. P.; Townsend, C. A.; Lamichhane, G.; Bianchet, M. A. Loss of a Functionally and Structurally Distinct LD-Transpeptidase, Ldt_{Mt5}, Compromises Cell Wall Integrity in Mycobacterium Tuberculosis. J. Biol. Chem. 2015, 290 (42), 25670-25685.
Bharatam, P. V.; Arfeen, M.; Patel, N.; Jain, P.; Bhatia, S.; Chakraborti, A. K.; Khullar, S.; Gupta, V.; Mandal, S. K. Design, Synthesis, and Structural Analysis of Divalent NI Compounds and Identification of a New Electron-Donating Ligand. Chem. —A Eur. J. 2016, 22 (3), 1088-1096.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 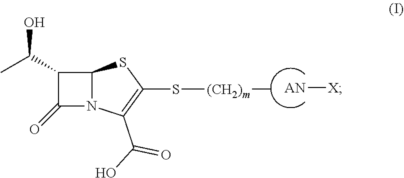
m is an integer selected from 0, 1, 2, 3, and 4;
A is a substituted or unsubstituted nitrogen-containing heterocyclic saturated ring;
X is selected from H, an alkylene chain, and a substituted or unsubstituted heterocyclic ring; and
stereoisomers, pharmaceutically acceptable salts thereof.

2. The compound of claim 1, wherein the compound of formula (I) comprises a compound of formula (I′): 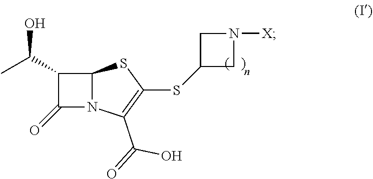
wherein n is an integer selected from 1, 2, and 3.

3. The compound of claim 1, wherein A is selected from azetidinyl, pyrrolidinyl, and piperidinyl.

4. The compound of claim 1, wherein X is selected from:

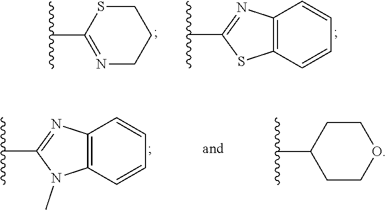

5. The compound of claim 1, wherein the compound of formula (I) is selected from:

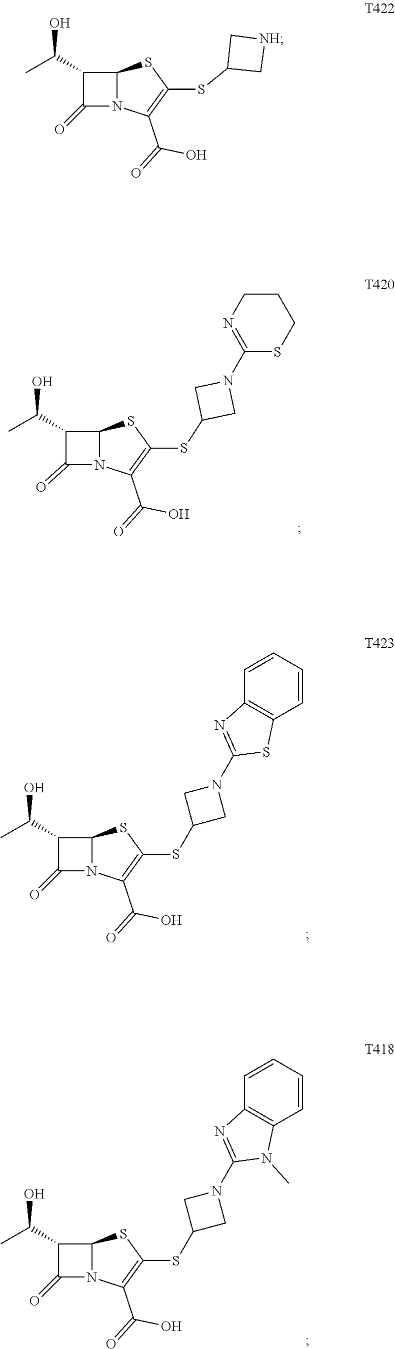 [SMILES: C[C@@H](O)[C@H]1C(=O)N2C(C(=O)O)=C(SC3CNC3)S[C@H]12 ; C[C@@H](O)[C@H]1C(=O)N2C(C(=O)O)=C(SC3CN(C4=NCCCS4)C3)S[C@H]12 ; C[C@@H](O)[C@H]1C(=O)N2C(C(=O)O)=C(SC3CN(c4nc5ccccc5s4)C3)S[C@H]12 ; C[C@@H](O)[C@H]1C(=O)N2C(C(=O)O)=C(SC3CN(c4nc5ccccc5n4C)C3)S[C@H]12]

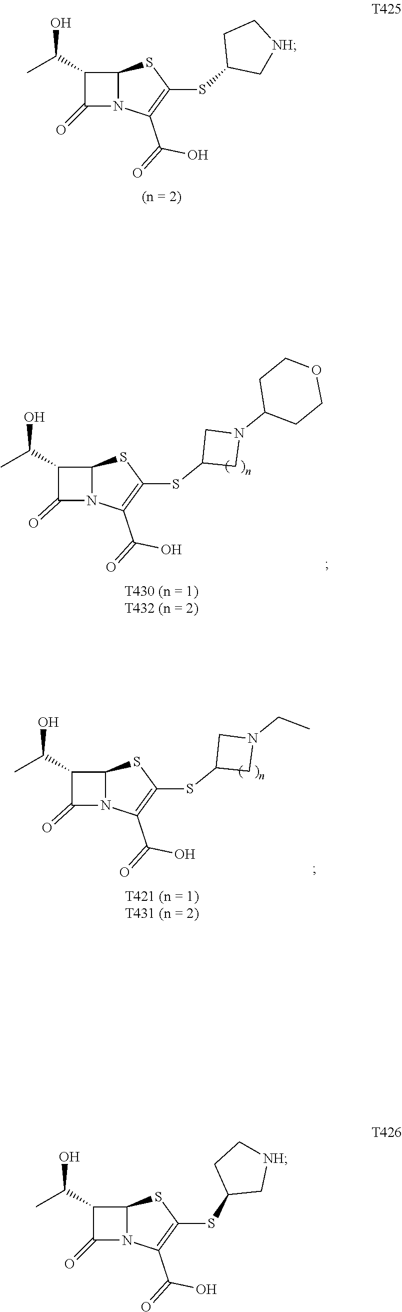

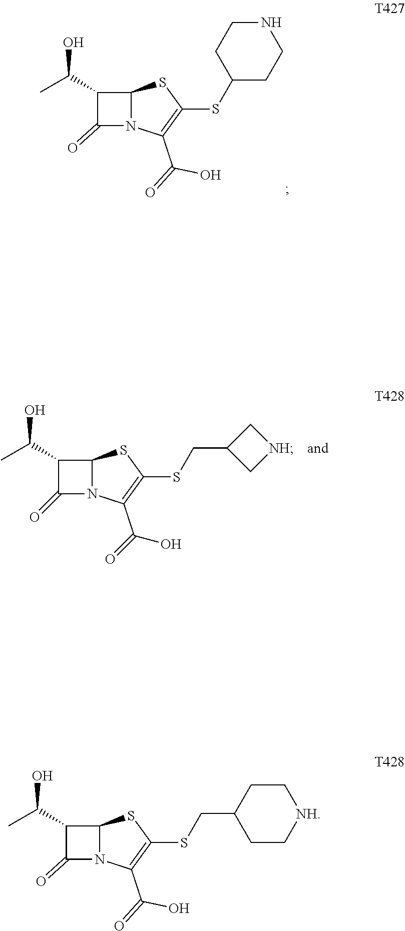 [SMILES: C[C@@H](O)[C@H]1C(=O)N2C(C(=O)O)=C(SC3CCNCC3)S[C@H]12 ; C[C@@H](O)[C@H]1C(=O)N2C(C(=O)O)=C(SCC3CNC3)S[C@H]12 ; C[C@@H](O)[C@H]1C(=O)N2C(C(=O)O)=C(SCC3CCNCC3)S[C@H]12]

6. A method for treating a mycobacterial infection in a subject, the method comprising administering a subject in need of treatment thereof, a therapeutically effective amount of a compound of formula (I) of claim 1.

7. The method of claim 6, wherein the mycobacterial infection is selected from a Mycobacterium tuberculosis infection, a nontuberculous mycobacterial infection, and a Mycobacteroides abscessus infection.

8. The method of claim 6, further comprising administering to the subject one or more antibiotics in combination with the compound of formula (I).

9. The method of claim 8, wherein the one or more antibiotics comprise one or more β-lactamase inhibitors.

10. The method of claim 8, wherein the one or more β-lactamase inhibitors is selected from clavulanate, sulbactam, tazobactam, and avibactam.

11. The method of claim 8, wherein the one or more β-lactamase inhibitors comprises clavulanate.

12. A pharmaceutically composition comprising a compound of formula (I) of claim 1 and one or more antibiotics.

13. The pharmaceutical composition of claim 12, wherein the one or more antibiotics comprises one or more β-lactamase inhibitors.

14. The pharmaceutical composition of claim 13, wherein the one or more β-lactamase inhibitors is selected from clavulanate, sulbactam, tazobactam, and avibactam.

15. The pharmaceutical composition of claim 14, wherein the one or more β-lactamase inhibitors comprises clavulanate.

16. A method for inhibiting an L,D-transpeptidase (Ldt) or a D,D-transpeptidase, the method comprising contacting the L,D-transpeptidase (Ldt) or D,D-transpeptidase with a compound of claim 1.

17. The method of claim 16, wherein the contacting is in vitro or in vivo.
